(19) United States
(12) Patent Application Publication
(10) Pub. No.: US 2024/0230677 A1
(43) Pub. Date: Jul. 11, 2024

(54) Protein Biomarkers For Non-Alcoholic Fatty Liver Disease (NAFLD)

(71) Applicant: Nederlandse Organisatie voor toegepast-natuurwetenschappelijk onderzoek TNO, 's-Gravenhage (NL)

(72) Inventors: Jan Roeland Occo Hanemaaijer, Voorhout (NL); Arianne Van Koppen, Bilthoven (NL); Lars Verschuren, De Meern (NL)

(21) Appl. No.: 18/562,985

(22) PCT Filed: May 31, 2022
(86) PCT No.: PCT/NL2022/050298

§ 371 Date: Nov. 21, 2023

(30) Foreign Application Priority Data
May 31, 2021 (EP) 21176833.8

(51) Int. Cl. G01N 33/68

(52) U.S. Cl. CPC G01N 33/6893; G01N 2800/085; G01N 2800/52; G01N 2800/60

(57) ABSTRACT

The invention relates to methods for typing and analysing blood, serum or plasma samples of subjects for the presence of protein biomarkers for non-alcoholic fatty liver disease (NAFLD), and to methods for classifying and diagnosing subjects on the basis of such protein biomarkers and/or for monitoring treatment of NAFLD.

### FIELD OF THE INVENTION

The invention relates to the field of non-alcoholic fatty liver disease (NAFLD). More specifically, the invention relates to methods for typing, classifying and diagnosing NAFLD. The invention further provides methods and means for differentiating different stages of NAFLD, in particular non-alcoholic steatohepatitis (NASH), based on protein biomarkers in blood, serum or plasma.

### BACKGROUND OF THE INVENTION

Non-alcoholic fatty liver disease (NAFLD), also referred to as metabolic-associated fatty liver disease (MAFLD), is a spectrum of chronic liver diseases, encompassing fatty liver, non-alcoholic steatohepatitis (NASH) and hepatic fibrosis. NAFLD affects 25% of the global adult population and is the most common chronic liver disease worldwide. Non-alcoholic steatohepatitis (NASH) is the advanced form of NAFLD, a chronic disease characterized by excessive fat accumulation in the liver, hepatic necroinflammation and fibrosis progression. NASH can progress to cirrhosis and subsequent hepatocellular carcinoma.

No therapies have been approved for NAFLD or NASH till date, although many clinical trials have been initiated. However, all of these have failed, many in late stages. Several reasons for failure to trials in cirrhotic patients have been suggested (Ratziu et al. 2020), including: 1) patient selection, especially the divergence between the target of a drug and the stage of the disease at which it is administered as early and late stages may not be driven by the same mechanisms or have the same therapeutic targets; and 2) insufficient diagnostic tools for classifying patients by disease stage.

Liver biopsy is currently the standard in NAFLD diagnosis. However, biopsy is an expensive and invasive procedure with a risk of complications, such as bleedings with associated morbidity and mortality, and shows variability in sampling. In addition, several non-invasive methods (such as MRI) are being developed to measure the elasticity of the liver, which is a measure of fibrosis (the more fibrosis, the lower tissue elasticity). But these methods are laborious, expensive, and not sensitive, and only patients with severe fibrosis can be identified. In addition, some blood-based markers exist, but they also are not sensitive, cannot discriminate between the various stages of disease, and are only suitable for diagnosis of late stage NASH or cirrhosis, but not for early stages. For example, tests based on plasma cytokeratin 18 (CK18) fragment levels (Cusi et al., 2014), soluble macrophage activation marker CD163 (Kazankov et al., 2016), a panel includes age, sex, AST, BMI, AST/ALT ratio, and serum hyaluronic acid (Palekar et al., 2006) have been described, but only with limited or late stage diagnostic value. Loomba et al. (2019) describe a serum test measuring alpha2 macroglobulin (A2M), hyaluronic acid (HA), and TIMP metallopeptidase inhibitor 1 (TIMP1), but this test also only discriminates patients with advanced fibrosis from those with mild to moderate fibrosis. Van Koppen et al. (2017) describe a gene expression signature for onset of NASH-related fibrosis in a NASH mouse model.

Current non-invasive methods and biomarkers are inadequate. Therefore in practice often a combination of methods is used, however with limited success and there is a great need for blood-based biomarkers.

With an increasing number of patients developing NASH-related end-stage liver disease (more than 3 million in the USA only) and pharmacological treatments on the horizon, there is a pressing need to develop NAFLD and NASH biomarkers for classification, assessment of therapeutic efficacy and selection of patients for treatment and monitoring. This requirement is particularly true as liver biopsy utility is limited by its invasive nature, poor patient acceptability and sampling variability. In particular, markers for early identification and classification of NASH are important, as this is key to successful prevention or and limitation of disease progression.

### SUMMARY OF THE INVENTION

It is an object of the present invention to provide novel and improved biomarkers for NAFLD, in particular NASH, and hepatic fibrosis that overcome one or more of the disadvantages described above. In particular, it is an object of the invention to provide specific, sensitive biomarkers for NAFLD and NASH that for use in non-invasive diagnosis of NAFLD and NASH.

The invention therefore provides a method for analysing a blood, serum or plasma sample of a subject, the method comprising determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D). In a preferred embodiment, the sample is a serum sample.

Also provided is a method for analysing a blood, serum or plasma sample of a subject, the method comprising determining the protein level of at least two proteins, wherein one of said at least two protein is selected from Insulin-like growth factor-binding protein 7 (IGFBP7), Urokinase-type plasminogen activator (uPA), Semaphorin 4D (SEMA4D), Versican (VCAN) and Scavenger Receptor Cysteine Rich Family Member With 5 Domains (SSC5D) and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2). Said at least two proteins are different. In a preferred embodiment, the sample is a serum sample.

In one embodiment, one of said at least two protein is selected from IGFBP7, SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, one of said at least two protein is selected from SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, said at least two proteins are selected from IGFBP7, SEMA4D and SSC5D.

In one preferred embodiment, a method of the invention comprises determining the protein levels of at least two protein selected from IGFBP7, SEMA4D and SSC5D.

In preferred embodiment, a method of the invention comprises determining the protein levels of SEMA4D and SSC5D.

In one preferred embodiment, a method of the invention comprises determining the protein levels of IGFBP7 and SSC5D.

In one preferred embodiment, a method of the invention comprises determining the protein levels of IGFBP7 and SEMA4D.

In one embodiment, a method of the invention comprises determining the protein levels of IGFBP7, SEMA4D and SSC5D.

In one embodiment, a method of the invention further comprises determining the protein levels of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10) and Annexin A3 (ANXA3).

In one embodiment, a method of the invention comprises determining the protein levels of IGFBP7, SEMA4D and SSC5D; IGFBP7, TNC and SSC5D; SEMA4D, uPA and TNC; IGFBP7, SSC5D and uPa; or IGFBP7, SSC5D and TNC.

In one embodiment, a method of the invention comprises determining the protein levels of IGFPB7, SSC5D, SEMA4D and uPA; SEMA4D, SSC5D, uPA and TNC; IGFBP7, SEMA4D, SSC5D and TNC; or IGFBP7, SEMA4D, uPA and TNC.

In one embodiment, a method of the invention comprises determining the protein levels of at least two proteins selected from IGFBP7, ANXA3, SEMA4D, FBN1, ADAMTS2, PAM and SSC5D.

In a further aspect, the invention provides a method for typing a blood, serum or plasma sample from subject, the method comprising determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) and typing said sample on the basis of said protein levels. In a preferred embodiment, the sample is a serum sample.

In one embodiment, a method of the invention comprises determining the protein levels of at least two proteins selected from IGFBP7, ANXA3, SEMA4D, FBN1, ADAMTS2, PAM and SSC5D.

Also provided is a method for typing a blood, serum or plasma sample from subject, the method comprising determining the protein level of at least two proteins, wherein one of said at least two protein is selected from Insulin-like growth factor-binding protein 7 (IGFBP7), Urokinase-type plasminogen activator (uPA), Semaphorin 4D (SEMA4D), Fibrillin 1(FBN1), Versican (VCAN), and Scavenger Receptor Cysteine Rich Family Member With 5 Domains (SSC5D) and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2), and typing said sample on the basis of said protein levels. Said at least two proteins are different.

In one embodiment, one of said at least two protein is selected from IGFBP7, SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, one of said at least two protein is selected from SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one preferred embodiment, a method of the invention comprises determining the protein levels of at least two protein selected from IGFBP7, SEMA4D and SSC5D and typing said sample on the basis of said protein levels.

In preferred embodiment, a method of the invention comprises determining the protein levels of SEMA4D and SSC5D and typing said sample on the basis of said protein levels.

In one preferred embodiment, a method of the invention comprises determining the protein levels of IGFBP7 and SSC5D and typing said sample on the basis of said protein levels.

In one preferred embodiment, a method of the invention comprises determining the protein levels of IGFBP7 and SEMA4D and typing said sample on the basis of said protein levels.

In one embodiment, a method of the invention comprises determining the protein levels of IGFBP7, SEMA4D and SSC5D and typing said sample on the basis of said protein levels.

In one embodiment, a method of the invention comprises determining the protein levels of IGFBP7, SEMA4D and SSC5D and typing said sample on the basis of said protein levels.

In one embodiment, a method of the invention further comprises determining the protein levels of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10) and Annexin A3 (ANXA3) and typing said sample on the basis of said protein levels.

In one embodiment, a method of the invention comprises determining the protein levels of IGFBP7, SEMA4D and SSC5D; IGFBP7, TNC and SSC5D; SEMA4D, uPA and TNC; IGFBP7, SSC5D and uPa; or IGFBP7, SSC5D and TNC and typing said sample on the basis of said protein levels.

In one embodiment, a method of the invention comprises determining the protein levels of IGFPB7, SSC5D, SEMA4D and uPA; SEMA4D, SSC5D, uPA and TNC; IGFBP7, SEMA4D, SSC5D and TNC; or IGFBP7, SEMA4D, uPA and TNC and typing said sample on the basis of said protein levels. In a further aspect, the invention provides a method for classifying a subject suffering from non-alcoholic fatty liver disease (NAFLD), preferably non-alcoholic steatohepatitis (NASH), or from hepatic fibrosis, or suspected of suffering therefrom, the method comprising determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of said subject and classifying the subject on the basis of said protein levels. In a preferred embodiment, the sample is a serum sample.

Also provided is a method for classifying a subject suffering from non-alcoholic fatty liver disease (NAFLD), preferably non-alcoholic steatohepatitis (NASH), or from hepatic fibrosis, or suspected of suffering therefrom, the method comprising determining the protein level of at least two proteins in a blood, serum or plasma sample of said subject, wherein one of said at least two protein is selected from Insulin-like growth factor-binding protein 7 (IGFBP7), Urokinase-35 type plasminogen activator (uPA), Semaphorin 4D (SEMA4D), Versican (VCAN), and Scavenger Receptor Cysteine Rich Family Member With 5 Domains (SSC5D), and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2), and classifying 40 the subject on the basis of said protein levels. Said at least two proteins are different.

In one embodiment, a method of the invention comprises determining the protein levels of at least two proteins selected from IGFBP7, ANXA3, SEMA4D, FBN1, ADAMTS2, PAM and SSC5D.

In one embodiment, one of said at least two protein is selected from IGFBP7, SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, one of said at least two protein is selected from SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one preferred embodiment, a method of the invention comprises determining the protein levels of at least two protein selected from IGFBP7, SEMA4D and SSC5D and classifying the subject on the basis of said protein levels.

In preferred embodiment, a method of the invention comprises determining the protein levels of SEMA4D and SSC5D and classifying the subject on the basis of said protein levels.

In one preferred embodiment, a method of the invention comprises determining the protein levels of IGFBP7 and SSC5D and classifying the subject on the basis of said protein levels.

In one preferred embodiment, a method of the invention comprises determining the protein levels of IGFBP7 and SEMA4D and classifying the subject on the basis of said protein levels.

In one embodiment, a method of the invention comprises determining the protein levels of IGFBP7, SEMA4D and SSC5D and classifying the subject on the basis of said protein levels.

In one embodiment, a method of the invention comprises determining the protein levels of IGFBP7, SEMA4D and SSC5D and classifying the subject on the basis of said protein levels.

In one embodiment, a method of the invention further comprises determining the protein levels of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10) and Annexin A3 (ANXA3) and classifying the subject on the basis of said protein levels.

In one embodiment, a method of the invention comprises determining the protein levels of IGFBP7, SEMA4D and SSC5D; IGFBP7, TNC and SSC5D; SEMA4D, uPA and TNC; IGFBP7, SSC5D and uPa; or IGFBP7, SSC5D and TNC and classifying the subject on the basis of said protein levels.

In one embodiment, a method of the invention comprises determining the protein levels of IGFPB7, SSC5D, SEMA4D and uPA; SEMA4D, SSC5D, uPA and TNC; IGFBP7, SEMA4D, SSC5D and TNC; or IGFBP7, SEMA4D, uPA and TNC and classifying the subject on the basis of said protein levels. In a further aspect, the invention provides a method for monitoring treatment of non-alcoholic fatty liver disease (NAFLD), preferably treatment of non-alcoholic steatohepatitis (NASH), and/or determining the efficacy of treatment of NAFLD, preferably treatment of NASH, in a subject, the method comprising:
determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of said subject at a first time point to provide a first value for the protein levels of said at least two proteins,
determining the protein level of said at least two proteins selected from IGFBP7, THBS1, TNC, CXCL10, uPA, ANXA3, SEMA4D, FBN1, VCAN, ADAMTS2, PAM and SSC5D in a blood, serum or plasma sample of said subject at a second time point to provide a second value for the protein levels of said at least two proteins,
comparing said first value and said second value to determine the efficacy of said treatment in said subject.

Also provided is a method for monitoring treatment of non-alcoholic fatty liver disease (NAFLD), preferably treatment of non-alcoholic steatohepatitis (NASH), and/or determining the efficacy of treatment of NAFLD, preferably treatment of NASH, in a subject, the method comprising:
determining the protein level of at least two proteins, wherein one of said at least two protein is selected from Insulin-like growth factor-binding protein 7 (IGFBP7), Urokinase-type plasminogen activator (uPA), Semaphorin 4D (SEMA4D), Versican (VCAN), and Scavenger Receptor Cysteine Rich Family Member With 5 Domains (SSC5D) and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2), in a blood, serum or plasma sample of said subject at a first time point to provide a first value for the protein levels of said at least one protein,
determining the protein level of said at least two proteins in a blood, serum or plasma sample of said subject at a second time point to provide a second value for the protein levels of said at least one protein,
comparing said first value and said second value to determine the efficacy of said treatment in said subject. Said at least two proteins are different.

In one embodiment, a method of the invention comprises determining the protein levels of IGFBP7 and SSC5D at said first and second time point and comparing said first value and said second value.

In one embodiment, one of said at least two protein is selected from IGFBP7, SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, one of said at least two protein is selected from SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, said at least two proteins are selected from IGFBP7, SEMA4D and SSC5D.

In one embodiment, a method of the invention comprises determining the protein levels of IGFBP7 and SSC5D at said first and second time point and comparing said first value and said second value.

In one embodiment, a method of the invention comprises determining the protein levels of IGFBP7, SEMA4D and SSC5D at said first and second time point and comparing said first value and said second value.

In one embodiment, a method of the invention further comprises determining the protein levels of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10) and Annexin A3 (ANXA3) at said first and second time point and comparing said first value and said second value.

In one embodiment, a method of the invention comprises determining the protein levels of IGFBP7, SEMA4D and SSC5D; IGFBP7, TNC and SSC5D; SEMA4D, uPA and TNC; IGFBP7, SSC5D and uPa; or IGFBP7, SSC5D and TNC at said first and second time point and comparing said first value and said second value.

In one embodiment, a method of the invention comprises determining the protein levels of IGFPB7, SSC5D, SEMA4D and uPA; SEMA4D, SSC5D, uPA and TNC; IGFBP7, SEMA4D, SSC5D and TNC; or IGFBP7, SEMA4D, uPA and TNC at said first and second time point and comparing said first value and said second value.

In a further aspect, the invention provides a method of classifying and treating a subject suffering from non-alcoholic fatty liver disease (NAFLD), preferably non-alcoholic steatohepatitis (NASH), or hepatic fibrosis, the method comprising:
determining the protein level of at least two protein selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of said subject,
comparing said determined protein levels or a value for said protein levels with a reference value, said reference value being the protein levels of the same proteins or a value for said protein levels in a blood, serum or plasma sample from healthy subjects, from subjects suffering from non-alcoholic fatty liver disease (NAFLD)), or from subjects suffering from NASH, in particular from subjects suffering from NASH fibrosis stage F0, F1, F2, F3 or F4,
classifying the subject as suffering from NAFLD, preferably NASH, or hepatic fibrosis and/or determining NASH severity, preferably determining NASH fibrotic stage; and
treating the subject classified as suffering from NAFLD, preferably NASH, or hepatic fibrosis and/or a particular NASH severity, preferably NASH fibrotic stage with vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-L-1023 (AngioLab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioglitazone hydrochloride, SGM-1019 (Second Genome), Semaglutide, Silymarin, TERN-101 (Terns), TVB-2640 (Sagimet Biosciences), Tesamorelin, Tipelukast, Tirzepatide, Tropifexor, Ursodeoxycholic acid berberine, VK-2809 (Viking Therapeutics), Vupanorsen, ARO-HSD (Arrowhead Pharmaceuticals), BIO89-100 (89bio), FT-4101 (FORMA Therapeutics), NN-6177 (Novo Nordisk), ZSP-1601 (Guangdong Raynovent Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDL-2965 (Indalo Therapeutics), ION-224 (Ionis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent, Biotech), AXA-1125 (Axcella Health), AXA-1957 (Axcella Health), ALN-HSD) (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist Therapeutics), RPC-8844 (Celgene), RT-200 (Renova Therapeutics), RT-210 (Renova Therapeutics), S-723595 (Shionogi), SC-410 (Sancilio), SCO)-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), liver transplantation or a combination thereof.

Also provided is a method of classifying and treating a subject suffering from non-alcoholic fatty liver disease (NAFLD), preferably non-alcoholic steatohepatitis (NASH), or hepatic fibrosis, the method comprising:
determining the protein level of at least two protein, wherein one of said at least two protein is selected from Insulin-like growth factor-binding protein 7 (IGFBP7), Urokinase-type plasminogen activator (uPA), Semaphorin 4D) (SEMA4D)), Versican (VCAN), and Scavenger Receptor Cysteine Rich Family Member With 5 Domains (SSC5D)) and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2), in a blood, serum or plasma sample of said subject,
comparing said determined protein levels or a value for said protein levels with a reference value, said reference value being the protein levels of the same proteins or a value for said protein levels in a blood, serum or plasma sample from healthy subjects, from subjects suffering from non-alcoholic fatty liver disease (NAFLD)), or from subjects suffering from NASH, in particular from subjects suffering from NASH fibrosis stage F0, F1, F2, F3 or F4,
classifying the subject as suffering from NAFLD, preferably NASH, or hepatic fibrosis and/or determining NASH fibrotic stage; and
treating the subject classified as suffering from NAFLD, preferably NASH, or hepatic fibrosis and/or a particular NASH fibrotic stage with one of the treatments listed herein above. Said at least two proteins are different.

In one embodiment, one of said at least two protein is selected from IGFBP7, SEMA4D and SSC5D and the other of said at least two protein is selected from IGFBP7, TNC, uPA, SEMA4D, VCAN, SSC5D), Protein Associated with MYC (PAM) and A disintegrin and metalloproteinase with thrombospondin motifs 2 (ADAMTS2).

In one embodiment, one of said at least two protein is selected from SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, said at least two proteins are selected from IGFBP7, SEMA4D and SSC5D.

In one embodiment, a method of the invention comprises determining the protein levels of IGFBP7 and SSC5D and classifying the subject on the basis of said protein levels.

In one embodiment, a method of the invention comprises determining the protein levels of IGFBP7, SEMA4D and SSC5D and classifying the subject on the basis of said protein levels and treating the subject.

In one embodiment, a method of the invention further comprises determining the protein levels of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10) and Annexin A3 (ANXA3) and classifying the subject on the basis of said protein levels and treating the subject.

In one embodiment, a method of the invention comprises determining the protein levels of IGFBP7, SEMA4D and SSC5D; IGFBP7, TNC and SSC5D; SEMA4D, uPA and TNC; IGFBP7, SSC5D and uPa; or IGFBP7, SSC5D and TNC and classifying the subject on the basis of said protein levels and treating the subject.

In one embodiment, a method of the invention comprises determining the protein levels of IGFPB7, SSC5D, SEMA4D and uPA; SEMA4D, SSC5D, uPA and TNC; IGFBP7, SEMA4D, SSC5D and TNC; or IGFBP7, SEMA4D, uPA and TNC and classifying the subject on the basis of said protein levels and treating the subject.

In a further aspect, the invention provides a method for assigning subjects to a clinical trial for treatment or prevention of non-alcoholic steatohepatitis (NASH), the method comprising classifying subjects as suffering from NAFLD, preferably NASH, or hepatic fibrosis and/or determining NASH severity, preferably determining NASH fibrotic stage with a method according to the invention and assigning subjects that are classified to said clinical trial.

In one embodiment, a method of the invention comprising determining the protein levels of at least two proteins selected from IGFBP7, ANXA3, SEMA4D, FBN1, ADAMTS2, PAM and SSC5D.

In a further aspect, the invention provides a kit of parts comprising means for determining protein level of at least two proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D (SEMA4D), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D).

In one embodiment, said kit of parts comprises means for determining protein level of at least two proteins selected from IGFBP7, ANXA3, SEMA4D, FBN1, ADAMTS2, PAM and SSC5D.

Also provided is a kit of parts comprising means for determining protein level of at least two proteins, wherein one of said at least two protein is selected from Insulin-like growth factor-binding protein 7 (IGFBP7), Urokinase-type plasminogen activator (uPA), Semaphorin 4D (SEMA4D), Versican (VCAN), and Scavenger Receptor Cysteine Rich Family Member With 5 Domains (SSC5D), and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2). Said at least two proteins are different.

In one embodiment, one of said at least two protein is selected from IGFBP7, SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, one of said at least two protein is selected from SEMA4D and SSC5D and the other of said at least two protein is selected from IGFBP7, TNC, uPA, SEMA4D, VCAN, SSC5D, Protein Associated with MYC (PAM) and A disintegrin and metalloproteinase with thrombospondin motifs 2 (ADAMTS2).

In one embodiment, said at least two proteins are selected from IGFBP7, SEMA4D and SSC5D.

In one embodiment, the kit comprises means for determining protein level of IGFBP7 and SSC5D.

In one embodiment, the kit comprises means for determining protein level of IGFBP7, SEMA4D and SSC5D.

In one embodiment, kit comprises means for determining protein level of IGFBP7, SEMA4D and SSC5D; IGFBP7, TNC and SSC5D; SEMA4D, uPA and TNC; IGFBP7, SSC5D and uPa; or IGFBP7, SSC5D and TNC.

In one embodiment, kit comprises means for determining protein level of IGFPB7, SSC5D, SEMA4D and uPA; SEMA4D, SSC5D, uPA and TNC; IGFBP7, SEMA4D, SSC5D and TNC; or IGFBP7, SEMA4D, uPA and TNC.

In one embodiment, the kit further comprises means for determining protein level of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10) and Annexin A3 (ANXA3).

### DETAILED DESCRIPTION

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (e.g. approximately 10, about 10) preferably means that the value may be the given value (e.g. 10) more or less 5% of the value.

The use of the alternative (e.g., “or”) should be understood to mean either one, both, or any combination thereof of the alternatives.

As used herein, the term “subject” encompasses humans and animals, preferably mammals. Preferably, a subject is a human.

The terms “treat” or “treatment” refer to inhibiting the disease or disorder, i.e., halting or reducing its development or at least one clinical symptom of the disease or disorder, and/or to relieving symptoms of the disease or condition. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

“Non-alcoholic fatty liver disease” or “NAFLD” refers to a spectrum of chronic liver diseases, encompassing fatty liver or non-alcoholic fatty liver (NAFL), to non-alcoholic steatohepatitis (NASH) with inflammation and different degrees of fibrosis to cirrhosis. NAFLD is also referred to as metabolic-associated fatty liver disease (MAFLD).

“Non-alcoholic fatty liver” or “NAFL” as used herein refers to early stage NAFLD, characterized by fatty liver, but an absence of inflammation and fibrosis.

“Fibrosis” as used herein refers to the formation of fibrous tissue in response to e.g. inflammation and is characterized by myofibroblast differentiation and deposition of matrix protein, including collagen.

“Hepatic fibrosis” as used herein refers to fibrosis present and/or occurring in the liver.

As used herein “progressive hepatic fibrosis” or “progressive fibrosis” refers to progression of the fibrotic process, i.e. an increase in fibrous tissue in the liver over time. In one embodiment, “progressive fibrosis” means that NASH is progressing to a more advanced fibrosis stage, e.g. F0is progressing to any of F1-F4, F1 is progressing to any of F2-F4, F2 is progressing to F3 or F4, or F3 is progressing to F4.

“Non-alcoholic steatohepatitis” or “NASH” refers to NAFLD wherein fat accumulation is associated with varying degrees of inflammation (hepatitis) and varying degrees of fibrosis in the liver.

“Cirrhosis” as used herein refers to a late stage of hepatic fibrosis where the liver is characterized by high accumulation of matrix proteins and loss of liver functionality.

“NAFLD or NASH severity” refers to severity of disease, e.g. distinguishing mild and severe disease. In one embodiment, NAFLD or NASH severity refers to the degree of fibrosis. The degree of fibrosis in NAFLD and NASH can be expressed in fibrosis stages, for example fibrosis stages F0, F1, F2, F3 and F4, whereby a higher number indicates a more advanced fibrosis stage and a lower number indicates a less advanced fibrosis stage. In particular, F0indicates NASH with inflammation but no fibrosis; F1 perisinusoidal or periportal fibrosis; F2 indicates perisinusoidal and portal/periportal fibrosis; F3 indicates bridging fibrosis (bridging perisinusoidal and portal/periportal fibrotic tissue); and F4 indicates cirrhosis (According to NASH CRB scoring system, as described in Kleiner et al. Hepatology 2005)). Alternatively, a distinction can be made between e.g. F0 vs F1-F2 vs F3-F4; no, mild and severe fibrosis, respectively, or e.g. between F0-F1, and F2 or F3-F4, between F0-F1 and F2-F3 or F4.

The term “analysing” as used herein refers to determining protein levels of indicated proteins in a blood, serum or plasma sample of a subject. Preferably, analysing comprises quantifying the protein levels, either absolutely or relative to a reference value. In a preferred embodiment, the sample is a serum sample.

The term “typing” as used herein refers to assessing the presence of NAFLD, in particular NASH, and/or determining disease activity or severity, e.g. staging of NAFLD, in particular NASH. Assessing the presence of NAFLD or NASH refers to distinguishing NAFLD or NASH from non-NAFLD or non-NASH. In one embodiment, typing preferably refers to differentiating NAFLD severity, in particular NASH severity, preferably NASH fibrosis stages, e.g. fibrosis stages F0, F1, F2, F3 and F4 or fibrosis stages F0, F1-F2 and F3-F4 or stages F0-F1, F2-F3 and F4 or stages F0-F1, F2 and F3-F4. Hence, typing as used herein preferably comprises determining the fibrosis stage (e.g. stages F0, F1, F2, F3 and F4 or stages F0, F1-F2 and F3-F4 or stages F0-F1, F2-F3 and F4 or stages F0-F1, F2 and F3-F4) of the subject suffering from NAFLD, in particular NASH, or from hepatic fibrosis from which the sample is derived. Methods of typing in accordance with the invention are in particular suitable for diagnoses of a subject suffering from NAFLD, in particular NASH, or from hepatic fibrosis from which the sample is derived.

The present inventors have identified a set of protein biomarkers that are useful in typing NAFLD. This set of biomarkers makes it possible in a minimal invasive way to diagnose patients with NASH and fibrosis without the need for liver biopsy. In addition, different stages of disease can be discriminated making it possible to a) treat only patients who need pharmaceutical intervention, and b) to stratify patient groups for clinical trials. Until now all clinical trials for new NASH/fibrosis drugs have failed, partly due to the great variability in patients.

The biomarkers were identified in a unique way, using a mix of transcriptomics, dynamic proteomics, translational animal model for fibrosis, patient transcriptomics data, patient blood serum data, and selection of suitable biomarker assays. The starting point was not to perform a phishing expedition (as often used for biomarkers), but use a novel approach of combining gene expression with the formation of new collagen, which is a hallmark of progressive fibrosis, in a translational animal model. By basing the biomarkers on newly formed collagen, biomarkers of progressive fibrosis, and thus progressive NASH, could be identified.

The upregulated genes were further analysed, and a final set of protein biomarkers was selected based on relevance in human samples and differential protein expression in blood samples. These protein biomarkers were validated in patient samples (tissue for gene expression and blood for protein expression) and using statistical tools an algorithm was defined for a selected number of biomarkers which together can be used as a diagnostic blood-based protein marker to diagnose in particular the activity, severity and/or fibrosis stage of NASH patients, based on the determination of the presence of progressive fibrosis.

As demonstrated in the Examples herein, in particular a combination of at least two different protein biomarkers with at least one of IGFBP7, uPA, SEMA4D, VCAN, and SSC5D and the other selected from IGFBP7, TNC, uPA, SEMA4D, VCAN, SSC5D, PAM and ADAMTS2, is useful in diagnosing NAFLD and NASH and in determining NAFLD activity severity, in particular in distinguishing NAFLD or NASH fibrosis stage, such as F0, F1-F2 and F3-F4 or in distinguishing NASH fibrosis stage F0, F1, F2, F3 and F4. It is demonstrated that in particular a combination of at least two biomarkers with one or two of IGFBP7, SEMA4D and SSC5D shows particularly good sensitivity and specificity in diagnosis and determining NAFLD activity or severity, in particular NAFLD or NASH fibrosis stage. Adding further protein biomarkers, such as TNC, uPA, VCAN, PAM and/or ADAMTS2, to the combination of IGFBP7, SEMA4D and/or SSC5D may further improve sensitivity and specificity (see FIGS. 8 and 9).

In a first aspect, the invention provides a method for analysing a blood, serum or plasma sample of a subject, the method comprising determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL.10), Urokinase-type plasminogen activator (uPA), Annexin A3 (ANXA3), Semaphorin 4D) (SEMA4D)), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D). Also provided is a method comprising determining the protein level of at least two proteins selected from IGFBP7, THBS1, TNC, CXCL10, uPA, ANXA3, SEMA4D, FBN1, VCAN, ADAMTS2, PAM and SSC5D in a blood, serum or plasma sample from a subject. Preferably, protein levels are quantified. Provided is therefore a method of quantifying protein levels in a blood, serum or plasma sample comprising determining the protein level of at least two proteins selected from IGFBP7, THBS1, TNC, CXCL10, uPA, ANXA3, SEMA4D, FBN1, VCAN, ADAMTS2, PAM and SSC5D. In a further aspect, the invention provides a method for typing a blood, serum or plasma sample from a subject, the method comprising determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL.10), Urokinase-type plasminogen activator (UPA), Annexin A3 (ANXA3), Semaphorin 4D) (SEMA4D)), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D)) and typing said sample on the basis of said protein level. In a preferred embodiment, one of said at least two protein is selected from IGFBP7, uPA, SEMA4D, VCAN, and SSC5D, and the other of said at least two protein is selected from IGFBP7, TNC, uPA, SEMA4D, VCAN, SSC5D, PAM and ADAMTS2. Said at least two proteins are different. In a preferred embodiment, the protein levels of at least one of IGFBP7, SEMA4D and SSC5D and one or IGFBP7, TNC, uPA, SEMA4D, VCAN, SSC5D), PAM and ADAMTS2 are determined and preferably quantified. In a further preferred embodiment, the protein levels of IGFBP7, TNC, uPA, SEMA4D, VCAN, SSC5D, PAM, ADAMTS2, THBS1, CXCL10 and/or ANXA3 are further determined and preferably quantified. In a further preferred embodiment, the protein levels of at least two proteins are selected from IGFBP7, SEMA4D and SSC5D are determined and preferably quantified. In a further preferred embodiment, the protein levels of IGFBP7, SEMA4D and SSC5D are determined and preferably quantified. In a further preferred embodiment, the protein levels of one the combinations of protein as listed in FIGS. 8 and 9 having an Area under the Curve higher than 0.65, preferably higher than 0.70, are determined and preferably quantified.

A value for the protein levels of said at least two proteins is preferably compared with a value for the protein levels of the same proteins in a reference. In one embodiment, protein levels of said at least two proteins are preferably compared with protein levels of the same proteins in a reference. In a preferred embodiment, said subject is suffering from fatty liver disease, in particular from non-alcoholic fatty liver disease (NAFLD)) or NASH or suspected of suffering therefrom.

In a preferred embodiment, a method for analysing or typing a blood, serum or plasma sample of a subject of the invention comprises determining protein level of at least three proteins selected from IGFBP7, THBS1, TNC, CXCL10, uPA, ANXA3, SEMA4D, FBN1, VCAN, ADAMTS2, PAM and SSC5D in the sample, more preferably at least four proteins, more preferably at least five proteins, more preferably at least six proteins, more preferably at least seven proteins, more preferably at least eight proteins, more preferably at least nine proteins, more preferably at least ten proteins, more preferably at least eleven proteins selected from IGFBP7, THBS1, TNC, CXCL10, uPA, ANXA3, SEMA4D, FBN1, VCAN, ADAMTS2, PAM and SSC5D. In one embodiment, the method comprises determining protein level of IGFBP7, THBS1, TNC, CXCL10, uPA, ANXA3, SEMA4D, FBN1, VCAN, ADAMTS2, PAM and SSC5D. In one embodiment, the method comprises determining protein level of IGFBP7, THBS1, TNC, CXCL10, uPA, ANXA3, FBN1, VCAN, ADAMTS2, PAM and SSC5D.

In one embodiment, typing a blood, serum or plasma sample from a subject, preferably a subject suffering from NAFLD or suspected of suffering therefrom, distinguishes NAFLD from non-NAFLD, and/or distinguishes different stages of NAFLD or NASH disease activity or severity, such as NAFLD or NASH fibrosis stages (e.g. stages F0, F1, F2, F3 and F4 or stages F0, F1-F2 and F3-F4).

The biomarkers of the present inventions are suitable for diagnostic purposes, as well as for monitoring treatment of NAFLD, in particular NASH, including determining efficacy of treatment of NAFLD, in particular NASH. Hence, in one aspect, the invention provides methods for classifying subjects based on the set of protein biomarkers disclosed herein. The term “classifying” as used herein refers to classifying subjects based on the protein level of two or more proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D) (SEMA4D), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of the subject, preferably based on the protein levels of at least one of IGFBP7, uPA, SEMA4D, VCAN, and SSC5D and one of IGFBP7, TNC, uPA, SEMA4D, VCAN, SSC5D, PAM and ADAMTS2, more preferably based on the protein levels of one of IGFBP7, SEMA4D and SSC5D and one of IGFBP7, TNC, uPA, SEMA4D, VCAN, SSC5D, PAM and ADAMTS2. In one preferred embodiment, “classifying” as used herein refers to classifying subjects based on the protein level of at least one or two of IGFBP7, SEMA4D and SSC5D. In one preferred embodiment, “classifying” as used herein refers to classifying subjects based on the protein level of at least IGFBP7, SEMA4D and SSC5D. In a further preferred embodiment, “classifying” as used herein refers to classifying subjects based on the protein level of one the combinations of protein as listed in FIGS. 8 and 9 having an Area under the Curve higher than 0.65, preferably higher than 0.70.

In preferred embodiments, “classifying” refers to determining NAFLD activity or severity, in particular determining fibrosis stages, e.g. distinguishing stages F0, F1, F2, F3 and F4 or stages F0, F1-F2 and F3-F4 or stages F0-F1, F2-F3 and F4 or stages F0-F1, F2 and F3-F4.

The present set of protein biomarkers is further useful in determining whether or not a subject is suffering from NAFLD, in particular suffering from NASH, or hepatic fibrosis. Hence, in one embodiment, a method is provided for classifying a subject suffering from NAFLD, in particular NASH, or from hepatic fibrosis, preferably from NAFLD, in particular NASH, or suspected of suffering therefrom, the method comprising determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL10), Urokinase-type plasminogen activator (uPA), Annexin A3 (ANXA3), Semaphorin 4D) (SEMA4D)), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of said subject and classifying the subject on the basis of said protein levels. Said method is preferably a method of diagnosing a subject. In particular, in one embodiment, a subject is classified or diagnosed as suffering from NAFLD, in particular NASH, or from hepatic fibrosis, preferably NAFLD, in particular NASH, or not suffering from NAFLD, in particular NASH, or from hepatic fibrosis, preferably NAFLD, in particular NASH. With such method it is possible to determine whether or not the subject is suffering from NAFLD, in particular NASH, or from hepatic fibrosis, preferably NAFLD, in particular NASH. Hence, such method preferably comprises diagnosing said subject as suffering from NAFLD, in particular NASH, or from hepatic fibrosis, or not suffering from NAFLD, in particular NASH, or from hepatic fibrosis. Provided is therefore a method for determining whether or not a subject is suffering from NAFLD, in particular NASH, or from hepatic fibrosis, preferably from NAFLD, more preferably NASH, the method comprising determining the protein level of at least two proteins selected from IGFBP7, THBS1, TNC, CXCL10, uPA, ANXA3, SEMA4D, FBN1, VCAN, ADAMTS2, PAM and SSC5D in a blood, serum or plasma sample of said subject and classifying the subject on the basis of said protein level. Said NAFLD is preferably NASH. Said haptic fibrosis is preferably progressive hepatic fibrosis. Said subject is preferably suspected of suffering from NAFLD. In another embodiment, said subject is suspected of suffering from NASH. In a preferred embodiment, the protein levels of one of IGFBP7, uPA, SEMA4D, VCAN, and SSC5D and one of IGFBP7, TNC, uPA, SEMA4D, VCAN, SSC5D, PAM and ADAMTS2 are determined. In a further preferred embodiment, the protein levels of one of IGFBP7, SEMA4D and SSC5D and one of IGFBP7, TNC, uPA, SEMA4D, VCAN, SSC5D, PAM and ADAMTS2 are determined. In a further preferred embodiment, the protein levels of one or two of IGFBP7, SEMA4D and SSC5D are determined. In a further preferred embodiment, the protein levels of IGFBP7, SEMA4D and SSC5D are determined. In a further preferred embodiment, the protein levels of one the combinations of protein as listed in FIGS. 8 and 9 having an Area under the Curve higher than 0.65, preferably higher than 0.70, are determined.

Preferably, a value for the for the protein levels of said at least two protein is compared with a reference value, as described herein above. In one embodiment, the protein levels of said at least two protein is compared with a reference value.

In one embodiment, a subject is classified or diagnosed as suffering NAFLD, in particular NASH, or from hepatic fibrosis, preferably from NAFLD, in particular NASH, if:
the protein levels or a value for the protein levels of the at least two proteins, preferably comprising one or two or all of IGFBP7, SEMA4D and SSC5D, more preferably at least SSC5D or SEMA4D, in the sample of the subject is higher than the protein levels or a value for the protein levels of the same proteins in the blood, serum or plasma sample from healthy subject, preferably the average protein levels or a value for the average protein levels in samples of a plurality of subjects, or
the protein level or a value for the protein levels of the at least two proteins, preferably comprising one or two or all of IGFBP7, SEMA4D and SSC5D, more preferably at least SSC5D or SEMA4D, is similar to the protein levels or a value for the protein levels of the same proteins in the blood, serum or plasma sample from a subject suffering from NAFLD, preferably the average protein levels or a value for the average protein levels in samples of a plurality of subjects suffering from NAFLD.

In one embodiment, a subject is classified or diagnosed as suffering NASH if:
the protein level or a value for the protein levels of the at least two proteins, preferably comprising one or two or all of IGFBP7, SEMA4D and SSC5D, more preferably at least SSC5D or SEMA4D, is higher than the protein levels or a value for the protein levels of the same proteins in the blood, serum or plasma sample from a healthy subject or subject suffering from NAFL, preferably the average protein levels or a value for the average protein levels in samples of a plurality of healthy subjects or subjects suffering from NAFL, or
the protein level or a value for the protein levels of the at least two proteins, preferably comprising one or two or all of IGFBP7, SEMA4D and SSC5D, more preferably at least SSC5D or SEMA4D, is similar to the protein levels or a value for the protein levels of the same proteins in the blood, serum or plasma sample from a subject suffering from NASH, preferably the average protein levels or a value for the average protein levels in samples of a plurality of subjects suffering from NASH.

Preferably, in a method of the invention, the protein levels or a value for said protein levels of the at least two proteins, preferably comprising one or two or all of IGFBP7, SEMA4D and SSC5D, more preferably at least SSC5D or SEMA4D, is compared with a reference value and a subject is classified or diagnosed based on said comparison. Classifying and diagnosing are preferably performed based on a comparison of the protein levelss or a value for said protein levels in blood, serum or plasma of the subject that is classified or diagnosed in comparison to a reference value. Hence, in a preferred embodiment, a method of classifying a subject in accordance with the invention comprises comparing the protein level of said at least two proteins selected from IGFBP7, THBS1, TNC, CXCL10, uPA, ANXA3, SEMA4D, FBN1, VCAN, ADAMTS2, PAM and SSC5D) in a blood, serum or plasma sample of said subject with a reference value. In one embodiment, a value for the protein levels of the at least two proteins, preferably comprising one or two or all of IGFBP7, SEMA4D) and SSC5D, more preferably at least SSC5D or SEMA4D, is compared with a reference value. The term “reference” or “reference sample” as used herein refers to a sample, in particular a blood, serum or plasma sample, that comprises preferably proteins, from a healthy subject not suffering from NAFLD or from an subject that is known to suffer from NAFLD, NASH or hepatic fibrosis, and preferably also known to suffer from a particular NASH fibrosis stage, e.g. stages F0, F1, F2, F3 and F4 or stages F0, F1-F2 and F3-F4 or stages F0-F1, F2-F3 and F4 or stages F0-F1, F2 and F3-F4. In preferred embodiments, the reference sample is a sample from a subject or subjects that are known to suffer from fibrosis stage F0. The term “reference value” refers to a value determined for a reference sample. In preferred embodiments, the reference value is a value for a reference sample from a subject or subjects that are known to suffer from fibrosis stage F0. The reference sample is preferably the same type of sample as the sample of the subject to be classified. The reference value is preferably a value determined in the same type of sample of the subject to be classified. I.e. if protein levels are determined in a blood sample, the reference is preferably also a blood sample; if protein levels are determined in a serum sample, the reference is preferably also a serum sample; if protein levels are determined in a plasma sample, the reference is preferably also a plasma sample. In a preferred embodiment, the test sample and the reference sample are a serum sample. The reference can be a reference sample obtained from a single subject, and/or the reference value can be the value of the particular protein level in a sample of a single subject. It is, however, preferred, that the reference value is the average of the particular protein levels or a value for said protein levels in a plurality of subjects, i.e. a plurality of healthy subjects, or a plurality of subjects known to suffer from NAFLD (NAFL, NASH and, in the case of NASH, preferably from the same NASH fibrosis stage). Said plurality is for instance at least 5 subjects, at least 10 subjects, at least 20 subjects, at least 30 subjects, at least 50 subjects, at least 75 subjects, or at least 100 subjects.

Based on a comparison with the protein levels or a value for said protein levels of the at least two proteins, preferably comprising one or two or all of IGFBP7, SEMA4D) and SSC5D, more preferably at least SSC5D or SEMA4D, in the reference, it can be determined whether a subject is likely to suffer from NAFLD, in particular NASH, or from hepatic fibrosis, or is likely to suffer from a particular NAFLD or NASH severity and/or fibrosis stage.

For instance, it can be determined whether protein levels or a value for said protein levels in the sample is higher than or similar to a reference value. Whether or not protein levels or a value for said protein levels is higher than or similar to a reference value can be determined using statistical methods that are appropriate and well-known in the art, generally with a probability value of less than five percent chance of the change being due to random variation. It is well within the ability of a skilled person to determine the amount of increase or similarity that is considered significant. Preferably, “higher than” is at least 10, at least 20, at least 40, or at least 50% higher than the reference value. Preferably, “similar to” is at most 20% difference, more preferably at most 10% difference between protein level determined and the reference value.

The present set of protein biomarkers is also useful in determining severity of disease, in particular of NAFLD, such as determining the NAFLD or NASH fibrosis stage, in a subject suffering from NASH. Therefore, in one embodiment, a method for classifying a subject suffering from NAFLD, in particular NASH, or from hepatic fibrosis, preferably from NAFLD, in particular NASH, or suspected of suffering therefrom comprises determining activity or severity of disease, in particular NAFLD or NASH fibrotic stage. Said method is preferably a method of diagnosing a subject. Preferably it is determined whether the subject is suffering from NAFLD or NASH fibrosis stage F0, F1, F2, F3 or F4, or from NAFLD or NASH fibrosis stage F0, F1-F2 or F3-F4. Further provided is therefore also a method for determining whether a subject is suffering from NAFLD or NASH fibrosis stage F0, F1, F2, F3 or F4 or from NAFLD or NASH fibrosis stage F0, F1-F2 or F3-F4 or stages F0-F1, F2-F3 and F4 or stages F0-F1, F2 and F3-F4, the method comprising determining the protein level of at least two proteins selected from IGFBP7, THBS1, TNC, CXCL10, uPA, ANXA3, SEMA4D, FBN1, VCAN, ADAMTS2, PAM and SSC5D in a blood, serum or plasma sample of said subject. and determining fibrosis stage on the basis of said protein level. Preferably, a value for the protein levels of said at least two protein is compared with a reference value, as described herein below. In one embodiment, the protein levels of said at least two protein is compared with a reference value. Said subject is preferably suffering from NAFLD, more preferably from NASH. Said method preferably comprises determining the stage of non-alcoholic fatty liver disease (NAFLD) in a subject, the method comprising:
determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL.10), Urokinase-type plasminogen activator (uPA), Annexin A3 (ANXA3), Semaphorin 4D (SEMA4D), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D)in a blood, serum or plasma sample of said subject, and
comparing said protein levels or a value for said protein levels with a reference value to determine the stage of NAFLD in said subject, wherein said reference value is the protein level of the same proteins or a value for said protein levels in a blood, serum or plasma sample from healthy subjects, from subjects suffering from non-alcoholic fatty liver (NAFL), or from subjects suffering from NASH, in particular from subjects suffering from NASH with a known fibrosis stage F0, F1, F2, F3 or F4.

In a preferred embodiment, the protein levels of one of IGFBP7, uPA, SEMA4D, VCAN, and SSC5D and one of IGFBP7, TNC, uPA, SEMA4D, VCAN, SSC5D, PAM and ADAMTS2 are further determined. In a further preferred embodiment, the protein levels of one of IGFBP7, SEMA4D and SSC5D and one of IGFBP7, TNC, uPA, SEMA4D, VCAN, SSC5D, PAM and ADAMTS2 are determined. In a further preferred embodiment, the protein levels of one or two of IGFBP7, SEMA4D and SSC5D are determined. In a further preferred embodiment, the protein levels of IGFBP7, SEMA4D and SSC5D are determined. In a further preferred embodiment, the protein levels of one the combinations of protein as listed in FIGS. 8 and 9 having an Area under the Curve higher than 0.65, preferably higher than 0.70, are determined.

In one embodiment, a subject is classified or diagnosed as suffering from a particular NAFLD or NASH fibrosis stage, e.g. stage F0, F1-F2 or F3-F4, or stage F0, F1, F2, F3 or F4 or stage F0-F1, F2-F3 or F4 or stage F0-F1, F2 or F3-F4, if the protein levels or a value for said protein levels of the at least two proteins, preferably comprising one or two or all of IGFBP7, SEMA4D and SSC5D, more preferably at least SSC5D or SEMA4D, is similar to the protein level of the same proteins or a value for said protein levels in the blood, serum or plasma sample from a subject suffering from said particular NASH fibrosis stage, preferably the average protein levels or a value for the average protein levels in samples of a plurality of subjects suffering from a particular NASH fibrosis stage. I.e. a subject is classified or diagnosed as suffering from NASH fibrosis stage F0, F1, F2, F3 or F4, respectively if the protein levels or a value for said protein levels of the at least two proteins, preferably comprising one or two or all of IGFBP7, SEMA4D and SSC5D, more preferably at least SSC5D or SEMA4D, is similar to the protein level of the same proteins or a value for said protein levels in the blood, serum or plasma sample from a subject suffering from NASH fibrosis stage F0, F1, F2, F3 or F4, respectively, preferably the average protein levels or a value for said average protein levels in samples of a plurality of subjects suffering from NASH fibrosis stage F0, F1, F2, F3 or F4, respectively.

In one embodiment, a subject is classified or diagnosed as suffering from a particular NAFLD or NASH stage, e.g. fibrosis stage, e.g. stage F0, F1, F2, F3 or F4, or stage F0, F1-F2 or F3-F4 or stage F0-F1, F2-F3 or F4 or stage F0-F1, F2 or F3-F4, if the protein levels or a value for said protein levels of the at least two proteins, preferably comprising one or two or all of IGFBP7, SEMA4D and SSC5D, more preferably at least SSC5D or SEMA4D, is higher than the protein level of the same proteins or a value for said protein levels in the blood, serum or plasma sample from a subject suffering from a less advanced NASH (fibrosis) stage and/or lower than the protein levels or a value for said protein levels of the same proteins in the blood, serum or plasma sample from a subject suffering from a more advanced NASH (fibrosis) stage, preferably the average protein levels or a value for said average protein levels in samples of a plurality of subjects. This is in particular relevant for classifying or diagnosing a subject as suffering from NASH fibrosis stage F3 or F4. As an example, a subject can be classified or diagnosed as suffering from NASH fibrosis stage F3, if the protein levels or a value for said protein levels of the at least two proteins, preferably comprising one or two or all of IGFBP7, SEMA4D) and SSC5D, more preferably at least SSC5D or SEMA4D, is higher than the protein levels or a value for said protein levels of the same proteins in the blood, serum or plasma sample from a subject suffering from NASH fibrosis stage F2 and/or lower than the protein levels or a value for said protein levels of the same proteins in the blood, serum or plasma sample from a subject suffering from NASH fibrosis stage F4, preferably the average protein levels or a value for said average protein levels in samples of a plurality of subjects.

In one embodiment, in addition to the determined protein levels of the biomarkers disclosed herein, other parameters are taken into account in a method of classifying a subject of the invention, in particular included in the value for the protein levels. Examples of such parameters include the age, gender, body-mass-index (BMI), Alanine aminotransferase (ALT) level, aspartate aminotransferase (AST) level, hemoglobin A1C level, C-reactive protein level, plasma triglyceride level and plasma cholesterol level of the subject. Said parameters are for instance included in the value for the determined protein levels.

As another example, the difference or similarity between a value for the protein levels in a sample and a reference value can be determined by determining a correlation or ratio of the value for the protein levels in the sample and the reference value. For example, it can be determined whether the value for the protein levels in a sample correlates to the value for the protein level of the same proteins in a reference. Said correlations between the values for the protein levels in the subject's sample and the reference, can be used to produce an overall similarity score for the protein biomarkers used. A similarity score is a measure of the average correlation of a value for the protein levels of a set of proteins in a sample from a subject and a reference value. This correlation can be numerically expressed, e.g. using a correlation coefficient. Several correlation coefficients can be used. Preferred methods are parametric methods which assume a normal distribution of the data. Said correlation or similarity score can for instance be, but does not need to be, a numerical value between +1, indicative of a high correlation between the value for the protein levels in a sample of said subject and said reference, and −1, which is indicative of an inverse correlation. A threshold can be used to differentiate between samples classified as suffering from NALFD or NASH or a particular NAFLD or NASH activity or severity, e.g. a particular fibrosis stage or samples classified as not suffering from NAFLD or NASH or fibrosis stage F0. Said threshold is an arbitrary value that allows for discrimination between samples from subjects. If a similarity threshold value is employed, it is preferably set at a value at which an acceptable number of subjects with a positive classification for e.g. suffering from NAFLD or NASH or a particular fibrosis stage would score as false negatives, and an acceptable number of subjects with a negative classification for e.g. suffering from NAFLD or NASH or a particular fibrosis stage would score as false positives.

The specific reference or reference sample and threshold that is used in a method of the invention for classifying a subject, depends on the specific method, and a skilled person is well capable of identifying and using an appropriate reference samples and reference value. Suitable references samples and values are provided in more detail herein below. The levels of expression of the proteins in a reference and/or a value for these levels are preferably stored on a computer, or on computer-readable media, to be used in comparisons to the level of level data from a sample of subject that is typed, analyzed or classified in accordance with the present invention. The term “reference value” refers to the protein level or a value for said protein level of a particular protein or of particular proteins in a reference, in particular the protein levels or a value for said protein levels of two or more of IGFBP7, THBS1, TNC, CXCL10, uPA, ANXA3, SEMA4D, FBN1, VCAN, ADAMTS2, PAM and SSC5D, preferably at least one or two or all of IBFBP7, SEMA4D and SSC5D.

In one preferred embodiment, the reference value is the protein levels or a value for said protein levels of the same at least two proteins that are determined in the sample of the subject to be classified in a blood, serum or plasma sample from healthy subjects, from subjects suffering from non-alcoholic fatty liver disease (NAFLD)), or from subjects known to suffer from NASH, in particular from subjects known to suffer from a particular NAFLD or NASH fibrosis stage, e.g. stages F0, F1, F2, F3 or F4 or stage F0, F1-F2 or F3-F4 or stage F0-F1, F2-F3 or F4 or stage F0-F1, F2 or F3-F4.

A value for the protein levels of multiple proteins as described herein (e.g. at least two, at least 3, at least 4, at least 5, at least 6, at least 7, at least 8, at least 9, at least 10, at least 11 or 12 proteins) is preferably a weighted value. Similarly, the reference value is preferably a weighted value for the same protein levels in the reference sample. Such weighted value can be determined by a person skilled in the art using any suitable method or algorithm, including ensemble methods that use multiple learning algorithms to obtain a predictive value. These weighted values are then based on the protein levels for the subject to be tested and the protein levels in the reference sample. Non-limiting examples of suitable methods are random forest classification, gradient boosting, artificial neural networks (NN), Adaptive Synthetic Sampling Approach for Imbalanced Learning (ADASYN) and kernel principal component analysis (KPCA).

In one embodiment, a random forest classifier is determined for the protein levels that are determined in accordance with the present invention.

In one embodiment, an ADASYN classifier is determined for the protein levels that are determined in accordance with the present invention.

A skilled person is well capable of determining values for protein levels in a sample and reference values and suitable threshold values for the different combinations of protein biomarkers according to the invention with a method as described herein.

In one embodiment, in addition to determined protein levels, other parameters are taken into account in the weighted value. In one embodiment, one or more parameters selected from age, gender, body-mass-index (BMI), Alanine aminotransferase (ALT) level, aspartate aminotransferase (AST) level, hemoglobin A1C level, C-reactive protein level, plasma triglyceride level and plasma cholesterol level of the subject are taken into account in the weighted value.

In another embodiment, the weighted value is based solely on the determined protein levels, of the at least two, at least 3, at least 4, at least 5, at least 6, at least 7, at least 8, at least 9, at least 10, at least 11 or 12 protein biomarkers as described herein.

In one embodiment the value, preferably the weighted value, for the protein levels is a value for the protein levels of IGFBP7, THBS1, TNC, CXCL10, uPA, ANXA3, FBN1, VCAN, ADAMTS2, PAM and SSC5D.

In one embodiment the value, preferably the weighted value, for the protein levels is a value for the protein levels of one of IGFBP7, uPA, SEMA4D, VCAN, and SSC5D and one of IGFBP7, TNC, uPA, SEMA4D, VCAN, SSC5D, PAM and ADAMTS2.

In one embodiment the value, preferably the weighted value, for the protein levels is a value for the protein levels of one of IGFBP7, SEMA4D and SSC5D and one of IGFBP7, TNC, uPA, SEMA4D, VCAN, SSC5D, PAM and ADAMTS2.

In one embodiment the value, preferably the weighted value, for the protein levels is a value for the protein levels of one or two of IGFBP7, SEMA4D and SSC5D.

In one embodiment the value, preferably the weighted value, for the protein levels is a value for the protein levels of IGFBP7, SEMA4D and SSC5D.

The present set of protein biomarkers is also useful in determining efficacy of treatment of NAFLD. Because the biomarkers are suitable for detecting progressive fibrosis, it has become possible to determine over time whether progression of fibrosis is inhibited or halted. For this purpose, it is for instance possible to determine the protein levels of at least two of the biomarkers described herein before and after treatment, at different time points during treatment or before treatment and at different time point during treatment. Also provided is therefore a method for monitoring treatment of NAFLD, preferably treatment of NASH, and/or determining the efficacy of treatment of NAFLD, preferably treatment of NASH, in a subject, the method comprising:
determining the protein level of at least two proteins selected from Insulin-like growth factor-binding protein 7 (IGFBP7), Thrombospondin 1 (THBS1), Tenacin C (TNC), C-X-C motif chemokine ligand 10 (CXCL.10), Urokinase-type plasminogen activator (uPA), Annexin A3 (ANXA3), Semaphorin 4D) (SEMA4D)), Fibrillin 1(FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of said subject at a first time point to provide a first value for the protein levels of said at least two proteins,
determining the protein level of said at least two proteins selected from IGFBP7, THBS1, TNC, CXCL10, uPA, ANXA3, SEMA4D, FBN1, VCAN, ADAMTS2, PAM and SSC5D in a blood, serum or plasma sample of said subject at a second time point to provide a second value for the protein levels of said at least two proteins,
comparing said first value and said second value to determine the efficacy of said treatment in said subject.

The method is suitable to monitor any treatment or determine efficacy of NAFLD, in particular NASH. Examples of such treatment are life style changes, treatment with vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-L-1023 (Angiolab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioglitazone hydrochloride, SGM-1019 (Second Genome), Semaglutide, Silymarin, TERN-101 (Terns), TVB-2640 (Sagimet Biosciences), Tesamorelin, Tipelukast, Tirzepatide, Tropifexor, Ursodeoxycholic acid berberine, VK-2809 (Viking Therapeutics), Vupanorsen, ARO-HSD (Arrowhead Pharmaceuticals), BIO89-100 (89bio), FT-4101 (FORMA Therapeutics), NN-6177 (Novo Nordisk), ZSP-1601 (Guangdong Raynovent Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDL-2965 (Indalo Therapeutics), ION-224 (Ionis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent Biotech), AXA-1125 (Axcella Health), AXA-1957 (Axcella Health), ALN-HSD) (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist Therapeutics), RPC-8844 (Celgene), RT-200 (Renova Therapeutics), RT-210 (Renova Therapeutics), S-723595 (Shionogi), SC-410 (Sancilio), SCO)-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), liver transplantation or a combination thereof.

In one embodiment, said first time point is prior to treatment and said second time point is after treatment. In another embodiment, said first time point is prior to treatment and said second time point is during treatment. In another embodiment, said first time point and said second time point are both during treatment, but at different time points. In another embodiment, said first time point is during treatment and said second time point is after treatment.

In one embodiment, the protein levels of at least two of the proteins is further determined at one or more further time points, e.g. at a third, fourth, fifth, sixth, seventh, etc. time point to provide one or more further values, e.g. a third, fourth, fifth, sixth, seventh, etc. value. These one or more further time points are preferably time points during treatment. This way the efficacy of the treatment can be closely monitored.

It is preferred that protein levels of the same at least two proteins are determined at the first and second time point and optional further time points.

In one embodiment, said first value and/or said second value and/or one or more optional further values are compared with one or more reference values. Said one or more reference values are preferably each a value for the protein levels of the same at least two proteins in a blood, serum or plasma sample from a healthy subject or subjects, from a subject or subjects suffering from NAFLD, or from a subject or subjects suffering from NASH, or from a subject or subjects suffering from a particular NASH fibrosis stage (e.g. from stage F0, F1, F2, F3 or F4 or from stage F0, F1-F2 or F3-F4 or stage F0-F1, F2-F3 or F4 or stage F0-F1, F2 or F3-F4).

In one embodiment, in addition to the determined protein levels of the biomarkers disclosed herein, other parameters are taken into account in a method for monitoring treatment of NAFLD, preferably treatment of NASH, and/or determining the efficacy of treatment of non-alcoholic fatty liver disease (NAFLD), preferably treatment of non-alcoholic steatohepatitis (NASH), of the invention, in particular included in the value for the protein levels. Examples of such parameters include the age, gender, body-mass-index (BMI), Alanine aminotransferase (ALT) level, aspartate aminotransferase (AST) level, hemoglobin A1C level, C-reactive protein level, plasma triglyceride level and plasma cholesterol level of the subject. Said parameters are for instance included in the value for the determined protein levels.

In a method of the invention, the protein level of at least two proteins selected from IGFBP7, THBS1, TNC, CXCL10, uPA, ANXA3, SEMA4D, FBN1, VCAN, ADAMTS2, PAM and SSC5D is determined in a blood, serum or plasma sample. In some embodiments, methods disclosed herein comprise obtaining or providing a blood, serum or plasma sample from the subject. A blood sample of a subject can be obtained by any standard method, for instance by venous extraction. In some embodiments, methods disclosed herein comprise isolating protein, preferably total protein, from the blood, serum or plasma sample.

In a method of the invention, the protein level of at least two proteins selected from IGFBP7, THBS1, TNC, CXCL10, uPA, ANXA3, SEMA4D, FBN1, VCAN, ADAMTS2, PAM and SSC5D is determined.

Insulin-like growth factor-binding protein 7 (IGFBP7) is identified by UniProtKB reference number Q16270.

Thrombospondin 1 (THBS1) is identified by UniProtKB reference number P07996.

Biglycan (BGN) is identified by UniProtKB reference number P21810.

Tenacin C (TNC) is identified by UniProtKB reference number P24821.

C-X-C motif chemokine ligand 10 (CXCL10) is identified by UniProtKB reference number P02778.

Urokinase-type plasminogen activator (uPA) is identified by UniProtKB reference number P00749.

Annexin A3 (ANXA3) is identified by UniProtKB reference number P12429.

Semaphorin 4D (SEMA4D), is identified by UniProtKB reference number Q92854.

Fibrillin 1 (FBN1) is identified by UniProtKB reference number P35555.

Versican (VCAN) is identified by UniProtKB reference number P13611.

A disintegrin and metalloproteinase with thrombospondin motifs 2 (ADAMTS2) is identified by UniProtKB reference number O95450.

Protein Associated with MYC (PAM) is identified by UniProtKB reference number P19021.

Scavenger Receptor Cysteine Rich Family Member With 5 Domains (SSC5D)) is identified by UniProtKB reference number A1L4H1.

In a preferred embodiment, a method of the invention comprises determining protein level of at least three proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least four proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least five proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least six proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least seven proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least eight proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least nine proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of at least ten proteins selected from IGFBP7, THBS1, TNC, CXCL10, uPA, ANXA3, SEMA4D, FBN1, VCAN, ADAMTS2, PAM and SSC5D in the sample. In another preferred embodiment, a method of the invention comprises determining protein level of all of IGFBP7, THBS1, TNC, CXCL10, uPA, ANXA3, SEMA4D, FBN1, VCAN, ADAMTS2, PAM and SSC5D in the sample.

In one preferred embodiment, a method of the invention comprises determining protein level of at least two, more preferably at least three, more preferably at least four, more preferably at least five, more preferably at least six, more preferably all, of the proteins selected from IGFBP7, ANXA3, SEMA4D, FBN1, ADAMTS2, PAM and SSC5D) in the sample.

In one preferred embodiment, no protein levels of proteins other than IGFBP7, THBS1, TNC, CXCL10, uPA, ANXA3, SEMA4D, FBN1, VCAN, ADAMTS2, PAM and SSC5D) are determined in a method of the invention.

In addition to the determined protein levels of the biomarkers disclosed herein, other parameters may be taken into account in a method of typing a typing a blood, serum or plasma sample of a subject or a method of classifying a subject of the invention, in particular included in the value for the protein levels. Examples of such parameters include the age, gender, body-mass-index (BMI), Alanine aminotransferase (ALT) level, aspartate aminotransferase (AST) level, hemoglobin A1C level, C-reactive protein level, plasma triglyceride level and plasma cholesterol level of the subject in typing the sample or classifying the subject.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least IGFBP7 and CXCL10; or at least IGFBP7 and uPA; or at least IGFBP7 and ANXA3; or at least IGFBP7 and SEMA4D; or at least IGFBP7 and FBN1; or at least IGFBP7 and VCAN; or at least IGFBP7 and ADAMTS2; or at least IGFBP7 and PAM; or at least IGFBP7 and SSC5D; or at least IGFBP7 and TNC; or at least IGFBP7 and THBS1; and optionally comprises determining the protein level of one or more other proteins selected from THBS1, TNC, CXCL10, uPA, ANXA3, SEMA4D, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least CXCL10 and uPA; or at least CXCL10 and ANXA3; or at least CXCL10 and SEMA4D; or at least CXCL10 and FBN1; or at least CXCL10 and VCAN; or at least CXCL10 and ADAMTS2; or at least CXCL10 and PAM; or at least CXCL10 and SSC5D; or at least CXCL10 and TNC; or at least CXCL10 and THBS1; and optionally comprises determining the protein level of one or more other proteins selected from IGFBP7, THBS1, TNC, uPA, ANXA3, SEMA4D, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least uPA and CXCL10; or at least uPA and THBS1; or at least uPA and ANXA3; or at least uPA and SEMA4D; or at least uPA and FBN1; or at least uPA and VCAN; or at least uPA and ADAMTS2; or at least uPA and PAM; or at least uPA and SSC5D; or at least uPA and TNC; and optionally comprises determining the protein level of one or more other proteins selected from IGFBP7, THBS1, TNC, CXCL10, ANXA3, SEMA4D, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least ANXA3 and CXCL10; or at least ANXA3 and uPA; or at least ANXA3 and THBS1; or at least ANXA3 and SEMA4D; or at least ANXA3 and FBN1; or at least ANXA3 and VCAN; or at least ANXA3 and PAM; or at least ANXA3 and SSC5D; or at least ANXA3 and TNC; or at least ANXA3 and ADAMTS2; and optionally comprises determining the protein level of one or more other proteins selected from IGFBP7, THBS1, TNC, CXCL10, uPA, SEMA4D, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least SEMA4D and uPA; or at least SEMA4D and ANXA3; or at least SEMA4D and FBN1; or at least SEMA4D and VCAN; or at least SEMA4D and ADAMTS2; or at least SEMA4D and PAM; or at least SEMA4D and SSC5D; or at least SEMA4D and TNC; or at least SEMA4D and THBS1; or at least SEMA4D and CXCL10; and optionally comprises determining the protein level of one or more other proteins selected from IGFBP7, THBS1, TNC, CXCL10, uPA, ANXA3, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least SSC5D and CXCL10; or at least SSC5D and uPA; or at least SSC5D and ANXA3; or at least SSC5D and SEMA4D; or at least SSC5D and FBN1; or at least SSC5D and VCAN; or at least SSC5D and ADAMTS2; or at least SSC5D and PAM; or at least SSC5D and THBS1; or at least SSC5D and TNC; and optionally comprises determining the protein level of one or more other proteins selected from IGFBP7, THBS1, TNC, CXCL10, uPA, ANXA3, SEMA4D, FBN1, VCAN, ADAMTS2, and PAM, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject, suffering from NAFLD, in particular NASH, or from hepatic fibrosis of suspected of suffering therefrom, or a method for determining the efficacy of treatment of NAFLD, preferably treatment of NASH, comprises determining the protein level of at least THBS1 and CXCL10; or at least THBS1 and uPA; or at least THBS1 and ANXA3; or at least THBS1 and SEMA4D; or at least THBS1 and FBN1; or at least THBS1 and VCAN; or at least THBS1 and ADAMTS2; or at least THBS1 and PAM; or at least THBS1 and SSC5D; or at least THBS1 and TNC; and optionally comprises determining the protein level of one or more other proteins selected from IGFBP7, TNC, CXCL10, uPA, ANXA3, SEMA4D, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least TNC and CXCL10; or at least TNC and uPA; or at least, TNC and ANXA3; or at least TNC and SEMA4D; or at least TNC and FBN1; or at least TNC and VCAN; or at least, TNC and ADAMTS2; or at least TNC and PAM; or at least TNC and SSC5D; or at least TNC and THBS1; and optionally comprises determining the protein level of one or more other proteins selected from IGFBP7, THBS1, CXCL10, uPA, ANXA3, SEMA4D, FBN1, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject, suffering from NAFLD, in particular NASH, or from hepatic fibrosis of suspected of suffering therefrom, or a method for determining the efficacy of treatment of NAFLD, preferably treatment of NASH, comprises determining the protein level of at least FBN1 and CXCL10; or at least FBN1 and uPA; or at least FBN1 and ANXA3; or at least FBN1 and SEMA4D; or at least FBN1 and THBS1; or at least FBN1 and VCAN; or at least FBN1 and ADAMTS2; or at least FBN1 and SSC5D; or at least FBN1 and TNC; and optionally comprises determining the protein level of one or more other proteins selected from IGFBP7, THBS1, TNC, CXCL10, uPA, ANXA3, SEMA4D, VCAN, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least VCAN and CXCL10; or at least VCAN and uPA; or at least VCAN and ANXA3; or at least VCAN and SEMA4D; or at least VCAN and FBN1; or at least VCAN and THBS1; or at least VCAN and ADAMTS2; or at least VCAN and PAM; or at least VCAN and SSC5D; or at least VCAN and TNC; and optionally comprises determining the protein level of one or more other proteins selected from IGFBP7, THBS1, TNC, CXCL10, uPA, ANXA3, SEMA4D, FBN1, ADAMTS2, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of at least ADAMTS2 and CXCL10; or at least ADAMTS2 and uPA; or at least ADAMTS2 and ANXA3; or at least ADAMTS2 and SEMA4D; or at least ADAMTS2 and FBN1; or at least ADAMTS2 and VCAN; or at least ADAMTS2 and PAM; or at least, ADAMTS2 and SSC5D; or at least ADAMTS2 and TNC; or at least ADAMTS2 and THBS1; and optionally comprises determining the protein level of one or more other proteins selected from IGFBP7, THBS1, TNC, CXCL10, uPA, ANXA3, SEMA4D, FBN1, VCAN, PAM and SSC5D, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of one of IGFBP7, uPA, SEMA4D, VCAN, and SSC5D and one of IGFBP7, TNC, uPA, SEMA4D, VCAN, SSC5D, PAM and ADAMTS2, and preferably comprises comparing said determined protein levels or a value for said protein levels with a reference value as disclosed herein.

In one embodiment, a method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of one of IGFBP7, SEMA4D and SSC5D and one of IGFBP7, TNC, uPA, SEMA4D, VCAN, SSC5D, PAM and ADAMTS2, and preferably comprises comparing said determined protein levels or a value for said protein levels with a reference value as disclosed herein.

A method of the invention for analysing a blood, serum or plasma sample of a subject, a method of the invention for typing a blood, serum or plasma sample from a subject, a method of the invention for classifying a subject suffering from NAFLD, in particular NASH, or from hepatic fibrosis of suspected of suffering therefrom, or a method for determining the efficacy of treatment of NAFLD, preferably treatment of NASH, comprises determining the protein level of one the combinations of protein as listed in FIGS. 8 and 9 having an Area under the Curve higher than 0.65, preferably higher than 0.70, and preferably comprises comparing said determined protein levels or a value for said protein levels with a reference value as disclosed herein.

In one embodiment, the subject is classified or diagnosed as suffering NAFLD, in particular NASH, or from hepatic fibrosis, in particular from NAFLD, if said determined protein levels are higher than the protein level of the same proteins in the blood, serum or plasma sample from healthy subject or healthy subjects, or the protein level of the at least two proteins, preferably comprising one or two or all of IGFBP7, SEMA4D and SSC5D, more preferably at least SSC5D or SEMA4D, is similar to the protein level of the same proteins in the blood, serum or plasma sample from a subject or subjects suffering from NAFLD.

In one embodiment, the subject is classified or diagnosed as suffering NASH if said determined protein levels are higher than the protein level of the same proteins in the blood, serum or plasma sample from a healthy subject or subjects or a subject or subjects suffering from NAFL, or similar to the protein level of the same proteins in the blood, serum or plasma sample from a subject or subjects suffering from NASH.

In one embodiment, the subject is classified or diagnosed as suffering from a particular NAFLD or NASH activity or severity, preferably a particular NAFLD or NASH fibrosis stage, e.g. stage F0, F1, F2, F3 or F4 or stage F0, F1-F2 or F3-F4 or stage F0-F1, F2-F3 or F4 or stage F0-F1, F2 or F3-F4, if the protein level of said determined proteins is similar to the protein level of the same proteins in the blood, serum or plasma sample from a subject or subjects suffering from a particular NAFLD or NASH activity or severity, preferably a particular NAFLD or NASH fibrosis stage, or higher than the protein level of the same proteins in the blood, serum or plasma sample from a subject or subjects suffering from a less advanced NAFLD or NASH severity, preferably a less advanced NAFLD or NASH fibrosis stage and/or lower than the protein level of the same proteins in the blood, serum or plasma sample from a subject or subjects suffering from a more advanced NAFLD or NASH activity or severity, preferably a more advanced NAFLD or NASH fibrosis stage.

In one preferred embodiment, a method for classifying a subject suffering from NAFLD, in particular NASH, or from hepatic fibrosis, preferably from NAFLD, in particular NASH, or suspected of suffering therefrom, and optionally comprising determining NAFLD or NASH activity or severity, in particular NAFLD or NASH fibrotic stage, comprises determining the protein level of at least two proteins selected from IGFBP7, CXCL10, uPA, ANXA3 and SSC5D in the sample, more preferably of at least three proteins selected from IGFBP7, CXCL10, uPA, ANXA3 and SSC5D, more preferably of at least four proteins selected from IGFBP7, CXCL10, uPA, ANXA3 and SSC5D), more preferably the protein levels of IGFBP7, CXCL10, uPA, ANXA3 and SSC5D. Preferably the protein levels of IGFBP7, CXCL10, and uPA, IGFBP7, CXCL10, and ANXA3, or IGFBP7, CXCL10, and SSC5D, or IGFBP7, uPA, and ANXA3, or IGFBP7, uPA, and SSC5D, or IGFBP7, ANXA3 and SSC5D, or CXCL10, uPA, and ANXA3, or CXCL10, uPA, and SSC5D, or uPA, ANXA3 and SSC5D, or IGFBP7, CXCL10, uPA, and ANXA3, or IGFBP7, uPA, ANXA3 and SSC5D, or IGFBP7, CXCL10, ANXA3 and SSC5D, or IGFBP7, CXCL10, uPA, and SSC5D, or CXCL10, uPA, ANXA3 and SSC5D, or IGFBP7, CXCL10, uPA, ANXA3 and SSC5D, or IGFBP7 and SSC5D, or IGFBP7 and SEM4D or SEMA4D and SSC5D) are determined. Optionally the method comprises determining the protein level of one or more other proteins selected from THBS1, TNC, SEMA4D, FBN1, VCAN, ADAMTS2, and PAM. Preferably the determined protein levels or a value for said protein levels is compared with a reference value. Said reference value is preferably a value for the protein levels of the same proteins in a blood, serum or plasma sample from a healthy subject or subjects, from a subject or subjects suffering from NAFLD, or from a subject or subjects suffering from NASH, or from a subject or subjects suffering from a particular NAFLD or NASH activity or severity, in particular NAFLD or NASH fibrosis stage (e.g. F0, F1, F2, F3 or F4 or stage F0, F1-F2 or F3-F4 or stage F0-F1, F2-F3 or F4 or stage F0-F1, F2 or F3-F4).

In one preferred embodiment, a method for monitoring treatment of NAFLD, preferably treatment of NASH, and/or determining the efficacy of treatment of NAFLD, preferably treatment of NASH, comprises determining the protein level of at least two proteins selected from THBS1, PAM, VCAN, ADAMTS2 and IGFBP7 in the sample, more preferably of at least three proteins selected from THBS1, PAM, VCAN, ADAMTS2 and IGFBP7, more preferably of at least four proteins selected from THBS1, PAM, VCAN, ADAMTS2 and IGFBP7, more preferably the protein levels of THBS1, PAM, VCAN, ADAMTS2 and IGFBP7. Optionally the method comprises determining the protein level of one or more other proteins selected from TNC, CXCL10, uPA, ANXA3, SEMA4D, FBN1, and SSC5D. Preferably the determined protein levels or a value for said protein levels is compared with a reference value. Said reference value is preferably a value for the protein levels of the same proteins in a blood, serum or plasma sample from a healthy subject or subjects, from a subject or subjects suffering from NAFLD, or from a subject or subjects suffering from NASH, or from a subject or subjects suffering from a particular NAFLD or NASH activity or severity, in particular NAFLD or NASH fibrosis stage (e.g. F0, F1, F2, F3 or F4 or stage F0, F1-F2 or F3-F4 or stage F0-F1, F2-F3 or F4 or stage F0-F1, F2 or F3-F4).

Methods for determining protein levels in a sample are well known in the art. A protein level in a blood, serum or plasma, preferably serum, may be determined by any assay known to a skilled person. Examples of such assays are polyacrylamide gel electrophoresis, including two dimensional gel electrophoresis, multidimensional protein identification technology, ELISA, bead-based immunoassays, immuno-PCR using, for example, Thunder-Link® antibody-oligonucleotide conjugation kit (Innova Biosciences. Cambridge UK), surface plasmon resonance, liquid chromatography -tandem mass spectrometry (LC-MS/MS), multiplex assay such as Luminex, meso scaled discovery (MSD) and matrix-assisted laser desorption/ionization time-of-flight mass spectrometry (MALDI-TOF). Further examples of suitable assays are chemo-luminescence assays, fluorescence assays, mass spectrometry, affinity chromatography, Western blotting, Northern blotting, histology and protein expression chips, probes. Preferred are multiplex systems that can measure expression levels from different proteins at the same time.

Mass spectrometry is a suitable means of determining a level of expression of a protein. A preferred method comprises liquid chromatography coupled to tandem mass spectrometry in positive electrospray ionization mode. The LC-MS/MS analysis may be performed, for example by using an I-Class UPLC system connected to a Xevo TQS mass spectrometer Waters (Manchester, UK), or an Q Exactive mass spectrometer (Thermo Fisher). A suitable multiplex system for determining a protein level is multiple reaction monitoring (MRM), which is a quantitative MS-based approach.

Said proteins are preferably detected and quantified using an immunochemical assay, preferably employing binding molecules such as antibodies that specifically bind to a ligand on said proteins. A protein is an antigen for an binding molecule that specifically reacts with said protein.

The binding molecules, preferably antibodies, are preferably coupled to a solid support such as a bead, monolithic material or a multi-well array. The binding molecules, preferably antibodies, may be coupled directly, or indirectly, for example by coupling of a second binding molecule that specifically recognizes the first binding molecule that binds to a protein. Indirect coupling may be accomplished, for example, by coupling of antibody-binding molecules such as protein A, protein G, or a mixture of protein A and G to beads, monolithic material or array. Direct coupling may be accomplished, for example, by cross-linking, covalently binding or physically adsorbing said binding molecule, preferably antibody, to the solid support.

A preferred method for determining a level of expression of a protein or multiple proteins includes Enzyme-Linked Immuno Sorbent Assay (ELISA) and Flow Cytometric ImmunoAssay (FCIA).

In a competition ELISA, known amounts of an antigen are immobilized to a surface. A sample comprising unknown amounts of said antigen is added, and the antigen is subsequently complexed with a binding molecule that is preferably conjugated, directly or indirectly, to a detectable label such as a colorimetric label, a fluorescent label, a radioactive label or a chemiluminescent label, or an enzyme. Following washing, detection of the binding molecule that is complexed to the immobilized antigen is accomplished by assessing the conjugated label or enzyme activity via incubation with a substrate to produce a measurable product. The amount of label or enzyme activity is inversely proportional to the amount of antigen in the sample.

A further preferred assay is a sandwich ELISA, in which a receptacle is coated with a first binding molecule that is specific to a protein, termed “capture binding molecule”, and detection of bound protein is accomplished with a second binding molecule, termed “detection binding molecule”. It is preferred that the capture and detection binding molecules do not interfere with each other and can bind simultaneously to said protein.

Said coating of a receptacle or bead, preferably the surface of a receptacle or bead, may be performed directly or indirectly. Indirect coating may be accomplished, for example, by using a biotin-labeled capture binding molecule that is attached to a linker molecule, for example a U-PLEX Linker (Meso-Scale Discovery, Rockville, USA). The employment of different linker molecules for different capture antibodies allows the generation of arrayed spots on a receptacle, each of which will bind to a specific protein. Said receptacle preferably is a multi-well plate, such as a 24 well plate, a 96 well plate, a 192 well plate, or a 384 well plate, in which each of the wells comprises arrayed spots, whereby each of the spots will bind to a specific protein.

Said second binding molecule is preferably directly or indirectly conjugated to a detectable label such as a colorimetric label, a fluorescent label a radioactive label, or a chemiluminescent label, or an enzyme. Detection of the amount of enzyme-conjugated binding molecule is preferably performed by incubation with a substrate to produce a measureable product. As an alternative, turbidimetric assays are preferred, especially for competition ELISAs.

Detectable labels are well known in the art. A detectable label may be a fluorescent, luminescent, chemiluminescent and/or electrochemiluminescent moiety which, when exposed to specific conditions, may be detected. For example, a fluorescent label may be exposed to radiation (i.e. light) at a specific wavelength and intensity to cause excitation of the fluorescent label, thereby enabling it to emit detectable fluorescence at a specific wavelength that may be detected.

Alternatively, the detectable label may be an enzyme which is capable of converting a (preferably undetectable) substrate into a detectable product that can be visualized and/or detected. Suitable enzymes include horseradish peroxidase, phosphatase, phosphatase/pyrophosphatase and luciferase.

Alternatively, the detectable label may be a radioactive label, which may be incorporated by methods known in the art.

Indirect labeling of a binding molecule may be accomplished, for example, through conjugation of a binding molecule with biotin and reacting biotin with labelled or enzyme-linked avidin or streptavidin.

As an alternative, carbon coated wells may be equipped with electrodes that produce chemical energy when subjected to an electrical charge, such as the Multi-array® and Multi-spot® 96-well plates of Meso-Scale Discovery. When combined with a SULFO-TAG® antibody, the chemical energy is transformed to emitted light which is measured using a high-resolution CCD camera.

The methods disclosed herein classify a subject as having NAFLD, in particular NASH, or specific fibrotic stages thereof. Preferably, the methods predict the likelihood that a subject is either suffering from or not suffering from NAFLD, in particular NASH, or from specific fibrotic stages thereof. Depending on the stage of the NAFLD and other factors, such as age, gender and BMI of the subject and other health conditions, treatment options can include life style changes, liver transplantation or treatment with active agents, such as vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-L-1023 (AngioLab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioglitazone hydrochloride, SGM-1019 (Second Genome), Semaglutide, Silymarin, TERN-101 (Terns), TVB-2640 (Sagimet Biosciences), Tesamorelin, Tipelukast, Tirzepatide, Tropifexor, Ursodeoxycholic acid berberine, VK-2809 (Viking Therapeutics), Vupanorsen, AO-HSD (Arrowhead Pharmaceuticals), BIO89-100 (89bio), FT-4101 (FORMA Therapeutics), NN-6177 (Novo Nordisk), ZSP-1601 (Guangdong Raynovent Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDL-2965 (Indalo Therapeutics), ION-224 (Ionis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent, Biotech), AXA-1125 (Axcella Health), AXA-1957 (Axcella Health), ALN-HSD) (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist Therapeutics), RPC-8844 (Celgene), RT-200 (Renova Therapeutics), RT-210 (Renova Therapeutics), S-723595 (Shionogi), SC-410 (Sancilio), SCO)-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), or a combination thereof. Accordingly, also provided is a method for determining a treatment schedule for a subject, comprising determining, using a method according to the invention as disclosed herein, whether a subject has NAFLD, in particular NASH, or hepatic fibrosis. If it. is determined that the individual has NAFLD, in particular NASH, or hepatic fibrosis, it can be determined if and how the individual can be treated. Preferably, such treatment comprises life style changes, liver transplantation or treatment with active agents, such as vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat, and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-1-1023 (AngioLab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioglitazone hydrochloride, SGM-1019 (Second Genome), Semaglutide, Silymarin, TERN-101 (Terns), TVB-2640 (Sagimet Biosciences), Tesamorelin, Tipelukast, Tirzepatide, Tropifexor, Ursodeoxycholic acid berberine, VK-2809 (Viking Therapeutics), Vupanorsen, ARO)-HSD (Arrowhead Pharmaceuticals), BIO89-100 (89bio), FT-4101 (FORMA Therapeutics), NN-6177 (Novo Nordisk), ZSP-1601 (Guangdong Raynovent, Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDI-2965 (Indalo Therapeutics), ION-224 (Ionis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent, Biotech), AXA-1125 (Axcella Health), AXA-1957 (Axcella Health), ALN-HSD) (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist, Therapeutics), RPC-8844 (Celgene), RT-200 (Renova Therapeutics), RT-210 (Renova Therapeutics), S-723595 (Shionogi), SC-410 (Sancilio), SCO)-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), or a combination thereof.

Provided is therefore, a method for determining a treatment strategy for a subject, comprising determining, using a method according to the invention as disclosed herein, whether a subject has NAFLD, in particular NASH, or hepatic fibrosis, and determining a treatment strategy for said subject if the determined protein levels indicates that said subject has NAFLD, in particular NASH, or hepatic fibrosis.

Also provided is a method of treatment of a subject in need thereof, comprising classifying the subject as having NAFLD, in particular NASH, or hepatic fibrosis, with a method according to the invention and providing said subject with treatment if the subject is classified as having NAFLD, in particular NASH, or hepatic fibrosis. Preferably, such treatment comprises life style changes, liver transplantation or treatment with active agents, such as vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-L-1023 (AngioLab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioglitazone hydrochloride, SGM-1019 (Second Genome), Semaglutide, Silymarin, TERN-101 (Terns), TVB-2640 (Sagimet, Biosciences), Tesamorelin, Tipelukast, Tirzepatide, Tropifexor, Ursodeoxycholic acid berberine, VK-2809 (Viking Therapeutics), Vupanorsen, ARO-HSD (Arrowhead Pharmaceuticals), BIO89-100 (89bio), FT-4101 (FORMA Therapeutics), NN-6177 (Novo Nordisk), ZSP-1601 (Guangdong Raynovent, Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDI-2965 (Indalo Therapeutics), ION-224 (Ionis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent Biotech), AXA-1125 (Axcella Health), AXA-1957 (Axcella Health), ALN-HSD) (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist Therapeutics), RPC-8844 (Celgene), RT-200 (Renova Therapeutics), RT-210 (Renova Therapeutics), S-723595 (Shionogi), SC-410 (Sancilio), SCO)-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), or a combination thereof.

Also provided is a method of classifying and treating a subject suffering from non-alcoholic fatty liver disease (NAFLD), preferably non-alcoholic steatohepatitis (NASH), or hepatic fibrosis, the method comprising:
determining the protein level of at least two protein selected from Insulin-like growth factor-binding protein 7 (IGFBP7), Thrombospondin 1 (THBS1), Tenacin C (TNC), C-X-C motif chemokine ligand 10 (CXCI.10), Urokinase-type plasminogen activator (uPA), Annexin A3 (ANXA3), Semaphorin 4D) (SEMA4D)), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D) in a blood, serum or plasma sample of said subject,
comparing said determined protein levels or a value for said protein levels with a reference value, said reference value being the protein levels of the same proteins or a value for said protein levels in a blood, serum or plasma sample from healthy subjects, from subjects suffering from non-alcoholic fatty liver (NAFL), or from subjects suffering from NASH, in particular from subjects suffering from NASH fibrosis stage F0, F1, F2, F3 or F4,
classifying the subject as suffering from NAFLD or hepatic fibrosis and/or determining NAFLD or NASH disease activity or severity, such as determining fibrotic stage; and
treating the subject classified as suffering from NAFLD, preferably NASH, or hepatic fibrosis and/or a specific NAFLD or NASH disease activity or severity, such as fibrotic stage, with vitamin E, metformin, pioglitazone, liraglutide, obeticholic acid, cenicriviroc, aramchol, Resmetirom, Semaglutide and other GLP receptor modulators, Tropifexor, Liraglutide, Nidufexor, Aldafermin, FGF21 mimick or analogs, growth hormone analogs, PPAR modulators, Firsocostat and other ACC modulators, Belapectin and other Galectin-3 inhibitors, EDP-305 and other bile acid receptor modulators, MSDC-0602K and other mTOT regulators, Selonsertib and other ASK1 inhibitors, Obeticholic acid, Aramchol, Cenicriviroc mesilate, Resmetirom, Selonsertib Hydrochloride, ALS-1-1023 (AngioLab), Aldafermin, Apararenone, BFKB-8488A Genentech), Belapectin, CC-90001 (Celgene), CRV-431 (Hepion Pharmaceuticals), Cenicriviroc/tropifexor, Cilofexor tromethamine, Cotadutide, EDP-305 (Enanta Pharmaceuticals), EYP-001 (Enyo Pharma), Efruxifermin, Elafibranor, Epeleuton, Ezetimibe, Firsocostat, GRI-0621 (Glyoregimmune), HHALPC (Promethera Biosciences), HM-15211 (Hanmi), Imm-124-E (Immuron), Icosabutate, LPCN-1144 (Lipocine), LYS-006 (Novartis), Lanifibranor, Leronlimab, Licogliflozin diprolinate, MET-409 (Metacrine), MSDC-0602K (Cirius Therapeutics), Metformin hydrochloride/sildenafil citrate/L-leucine, Naltrexone hydrochloride, Namodenoson, Nitazoxanide, Oramed insulin (Oramed), PF-05221304 (Pfizer), PF-06835919 (Pfizer), PF-06865571 (Pfizer), PXL-770 (Poxel), Peqbelfermin, Pemafibrate, Pioglitazone hydrochloride, SGM-1019 (Second Genome), Semaglutide, Silymarin, TERN-101 (Terns), TVB-2640 (Sagimet, Biosciences), Tesamorelin, Tipelukast, Tirzepatide, Tropifexor, Ursodeoxycholic acid berberine, VK-2809 (Viking Therapeutics), Vupanorsen, ARO-HSD (Arrowhead Pharmaceuticals), BIO89-100 (89bio), FT-4101 (FORMA Therapeutics), NN-6177 (Novo Nordisk), ZSP-1601 (Guangdong Raynovent, Biotech), 25-OH Cholesterol 3beta-sulfate, 5(R)-Deuteropioglitazone, AGN-242266 (Allergan), ALB-127158 (ConSynance Therapeutics), AZD-2693 (AstraZeneca), CB-4211 (CohBar), CER-209 (ABIONYX Pharma), CM-101 (ChemomAb), DWP-10292 (Daewoong), FM-101 (Future Medicine), Foralumab, GB-1211 (Galecto Biotech), HEC-96719 (HEC Pharma), HPG-1860 (Hepagene Therapeutics), IDL-2965 (Indalo Therapeutics), ION-224 (Ionis Pharmaceuticals), Miricorilant, NGM-395 (NGM Biopharmaceuticals), NN-9500 (Novo Nordisk), Nimacimab (Bird Rock Bio), PAT-409 (Blade Therapeutics), PBI-4547 (Liminal BioSciences), TERN-201 (Terns), Technetium Tc 99m tilmanocept, XW-003 (Hangzhou Sciwind Biosciences), XSP-0678 (Guangdong Raynovent, Biotech), AXA-1125 (Axcella Health), AXA-1957 (Axcella Health), ALN-HSD) (Alnylam Pharmaceuticals), ASC-41 (Ascletis), HPN-01 (Hepanova), AGN-242256 (Allergan), ALY-688 (Allysta Pharmaceuticals), ANG-3070 (Angion Biomedica), ASC-42 (Ascletis), Atrosab, Atrosimab, Bemitil, Bromantane, CJ-14199 (CH HealthCare), CRB-4001 (Corbus Pharmaceuticals), EDP-297 (Enanta Pharmaceuticals), GDD-3898 (Lipidio Pharma), Gel-B (Gelesis), Hu-6 (Rivus Pharmaceuticals), INB-03 (INmune Bio), JT-194 (Jecure Therapeutics), NGM-386 (NGM Biopharmaceuticals), NVP-018 (Abliva), Nicodicosapent, PRX-106 (Protalix Biotherapeutics), RLA-8 (Beijing Institute Pharmacology Toxicol), RLBN-1127 (Acquist Therapeutics), RPC-8844 (Celgene), RT-200 (Renova Therapeutics), RT-210 (Renova Therapeutics), S-723595 (Shionogi), SC-410 (Sancilio), SCO-116 (SCOHIA), SFA-001 (Sinew Pharma), SP-1373 (Altimmune), SRT-015 (Seal Rock Therapeutics), TERN-501 (Terns), URK-614 (ImmuPharma), YH-25724 (Yuhan), liver transplantation, life style changes, or a combination thereof.

As described herein above, no therapies have been approved for NAFLD or NASH, or hepatic fibrosis till date, although many clinical trials have been initiated, but all of these have failed, many in late stages. One reason for this failure is that until now it has been difficult to accurately determine disease activity or severity, such as fibrosis stage, in NASH patients and thus to classify patients in clinical trials by disease activity or severity, e.g. disease stage. Now that the present invention makes it possible to accurately diagnose NAFLD patients, to accurately determine the presence of progressive fibrosis and to accurately determine disease activity or severity, such as fibrosis stage, in NASH patients, it has become possible to assign NAFLD patient to clinical trials and classify patient therein, based on e.g. NAFLD or NASH disease activity or severity, e.g. fibrotic stage, and the presence or absence of hepatic fibrosis and/or progressive fibrosis. The invention therefore also provides a method for assigning subjects to a clinical trial for treatment or prevention of non-alcoholic steatohepatitis (NASH), the method comprising classifying subjects as suffering from NAFLD or hepatic fibrosis and/or determining NAFLD or NASH disease activity or severity, such as fibrotic stage with a method according to the invention and assigning subjects that are classified to said clinical trial. In a preferred embodiment, said method comprises determining protein level of at least three proteins selected from IGFBP7, THBS1, TNC, CXCL10, uPA, ANXA3, SEMA4D, FBN1, VCAN, ADAMTS2, PAM and SSC5D in the sample, more preferably at least four proteins, more preferably at least five proteins, more preferably at least six proteins, more preferably at least seven proteins, more preferably at least eight proteins, more preferably at least nine proteins, more preferably at least ten proteins selected from IGFBP7, THBS1, TNC, CXCL10, uPA, ANXA3, SEMA4D, FBN1, VCAN, ADAMTS2, PAM and SSC5D. In one embodiment, the method comprises determining protein level of IGFBP7, THBS1, TNC, CXCL10, uPA, ANXA3, SEMA4D, FBN1, VCAN, ADAMTS2, PAM and SSC5D).

Also provided is a kit of parts comprising means for determining protein level of at least two proteins selected from Insulin-like growth factor-binding protein 7 (IGFBP7), Thrombospondin 1 (THBS1), Tenacin C (TNC), C-X-C motif chemokine ligand 10 (CXCL.10), Urokinase-type plasminogen activator (uPA), Annexin A3 (ANXA3), Semaphorin 4D) (SEMA4D)), Fibrillin 1 (FBN1), Versican (VCAN), A disintegrin and metalloproteinase with thrombospondin motifs 2 (ADAMTS2), Protein Associated with MYC (PAM) and Scavenger Receptor Cysteine Rich Family Member With 5 Domains (SSC5D). Said kit is preferably for analysing a sample in accordance with the invention, for typing a sample in accordance with the invention and/or for classifying a subject in accordance with the invention. Said means are preferably suitable for determining protein level of the at least two proteins, preferably comprising one or two or all of IGFBP7, SEMA4D and SSC5D, more preferably at least SSC5D or SEMA4D, in a blood, serum or plasma sample. Such kit may comprise one or more of the following components: a container for collecting blood, serum or plasma, a container filled with preservative and/or one or more test tubes and other materials such as buffers and enzymes for analysis, and instructions for use.

The kit of parts according to the invention may be selected from any suitable assay and data processing apparatus and equipment, e.g. as described herein above. Said means for determining protein level of the at least two proteins, preferably comprising one or two or all of IGFBP7, SEMA4D and SSC5D, more preferably at least SSC5D or SEMA4D, preferably are means for an immunochemical assay. Said means for determining protein levels for instance comprise binding molecules such as antibodies that specifically bind to the at least two proteins, preferably comprising one or two or all of IGFBP7, SEMA4D and SSC5D, more preferably at least SSC5D or SEMA4D, or to an epitope in said proteins. The binding molecules, preferably antibodies, may be coupled directly, or indirectly, to a detectable label, such as a colorimetric label, a fluorescent label, a radioactive label or a chemiluminescent label, or an enzyme. Said detectable label is preferably a non-amino acid label, for instance a fluorescent label, a radioactive label or a chemiluminescent label, in particular a non-amino acid fluorescent, radioactive or chemiluminescent label. Indirect coupling to a label is for example by coupling of a second, labeled binding molecule, preferably antibody, that specifically recognizes the first binding molecule that binds to the at least two proteins, preferably comprising one or two or all of IGFBP7, SEMA4D and SSC5D, more preferably at least SSC5D or SEMA4D, or epitope of said proteins.

Said kit preferably comprises means for determining protein level of at least three proteins selected from IGFBP7, THBS1, TNC, CXCL10, uPA, ANXA3, SEMA4D, FBN1, VCAN, ADAMTS2, PAM and SSC5D, more preferably at least four proteins, more preferably at least five proteins, more preferably at least six proteins, more preferably at least seven proteins, more preferably at least eight proteins, more preferably at least nine proteins, more preferably at least ten proteins selected from IGFBP7, THBS1, TNC, CXCL10, uPA, ANXA3, SEMA4D, FBN1, VCAN, ADAMTS2, PAM and SSC5D. In one embodiment, the kit comprise means for determining protein level of IGFBP7, THBS1, TNC, CXCL10, uPA, ANXA3, SEMA4D, FBN1, VCAN, ADAMTS2, PAM and SSC5D.

In one embodiment, said kit of part comprises means for determining protein level of at least two proteins selected from IGFBP7, CXCL10, uPA, ANXA3 and SSC5D, more preferably means for determining protein level of at least three proteins selected from IGFBP7, CXCL10, uPA, ANXA3 and SSC5D, more preferably means for determining protein level of at least four proteins selected from IGFBP7, CXCL10, uPA, ANXA3 and SSC5D, more preferably means for determining protein level of at least IGFBP7, CXCL10, uPA, ANXA3 and SSC5D

In one embodiment, said kit or part comprises means for determining protein level of at least two proteins selected from THBS1, PAM, VCAN, ADAMTS2 and IGFBP7, more preferably means for determining protein level of at least three proteins selected from THBS1, PAM, VCAN, ADAMTS2 and IGFBP7, more preferably means for determining protein level of at least four proteins selected from THBS1, PAM, VCAN, ADAMTS2 and IGFBP7, more preferably means for determining protein level of at least THBS1, PAM, VCAN, ADAMTS2 and IGFBP7.

In a preferred embodiment, the kit comprises means for determining protein level of one of IGFBP7, uPA, SEMA4D, VCAN, and SSC5D and one of IGFBP7, TNC, uPA, SEMA4D, VCAN, SSC5D, PAM and ADAMTS2. In a further preferred embodiment, the kit further comprises means for determining protein level of one of IGFBP7, SEMA4D and SSC5D and one of IGFBP7, TNC, uPA, SEMA4D, VCAN, SSC5D, PAM and ADAMTS2. In a further preferred embodiment, kit comprises means for determining protein level of one or two of IGFBP7, SEMA4D and SSC5D. In a further preferred embodiment, the kit comprises means for determining protein level of IGFBP7, SEMA4D and SSC5D. In a further preferred embodiment, the kit comprises means for determining the protein level of one the combinations of protein as listed in FIGS. 8 and 9 having an Area under the Curve higher than 0.65, preferably higher than 0.70

Also provided is a kit of parts according to the invention for use in a method of analysing a sample in accordance with the invention, a method of typing a sample in accordance with the invention and/or a method for classifying a subject in accordance with the invention.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples. It will be understood that many variations in the methods described can be made without departing from the spirit of the invention and the scope of the appended claims.

### BRIEF DESCRIPTION OF THE DRAWINGS

FIG. 1: Sequence of activities on patient data generation. Numbers represent the number of samples which were used for the next step.

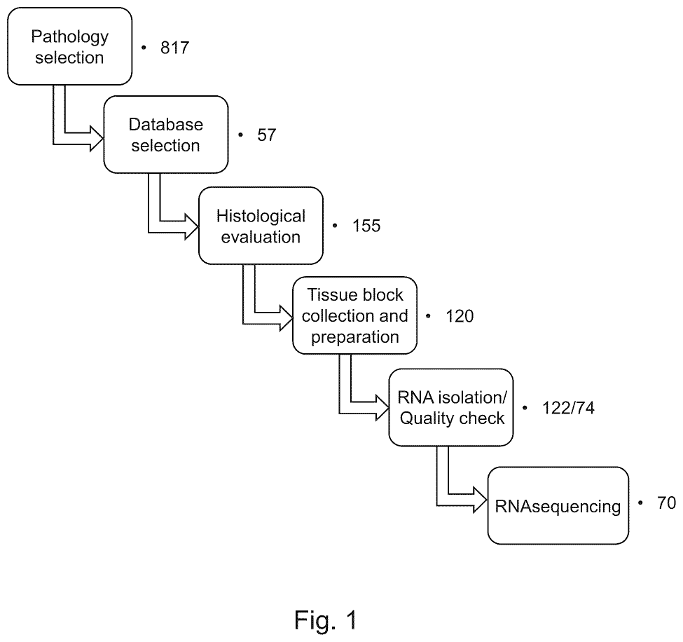

FIG. 2: Biomarker analysis in sera from NASH/fibrosis patients with different stages of fibrosis. CXCL10 (pg/ml), IGFBP1 (ng/ml), uPA (pg/ml), SSCD5 (ng/ml).

FIG. 3: Random forest confusion matrix predicting the stage of 29 patients with various stages of fibrosis, based on 11 of the 12 biomarkers analysed.

FIG. 4: Serum levels of a) uPA, b) IGFBP7, c) SSC5D, d) SEMA4D, e) TNC and f) VCAN in samples from NASH patients with biopsy-proven fibrosis score (F0-F4). Values represent mean +SEM for n=26 for F0, n=18 for F1, n=47 for F2, n=24 for F3, and n=28 for F4. * p <0.05, vs. F0samples.

FIG. 5: Prediction model using serum data from IGFBP7, SSC5D and SEMA4D. A) AUROC curve to show the predictive value of this set of biomarkers to distinguish the individual NAFLD fibrosis F-scores (F1-F2-F3-F4) as compared to reference F0; B) confusion matrix to show the predicted and true F-scores (reference); C) calculated sensitivity and specificity based on the predictions.

FIG. 6: Prediction model using serum data from IGFBP7, SSC5D and SEMA4D. A) AUROC curve to show the predictive value of this set of biomarkers to distinguish the grouped F-scores (mild, F1 and F2; advanced, F3 and F4) as compared to reference F0; B) confusion matrix to show the predicted and true F-scores (reference); C) calculated sensitivity and specificity based on the predictions.

FIG. 7: Prediction model using serum data from FBN1, VCAN and THBS1. A) AUROC curve to show the predictive value of this set of biomarkers to distinguish the grouped F-scores (mild, F1 and F2; advanced, F3 and F4) as compared to reference F0; B) confusion matrix to show the predicted and true F-scores (reference); C) calculated sensitivity and specificity based on the predictions.

FIG. 8: Comparison of prediction model using different combination of serum biomarker data. The Area Under the Curve (AUROC) shows the predictive value of each set of biomarkers to distinguish the individual NAFLD fibrosis F-scores (F1-F2-F3-F4) as compared to reference F0.

FIG. 9: Comparison of prediction model using different combination of serum biomarker data. The Area Under the Curve (AUROC) shows the predictive value of each set of biomarkers to distinguish the grouped F-scores (mild, F1 and F2; advanced, F3 and F4) as compared to reference F0.

### EXAMPLES

### 1. Methods

### Human Material Collection

Medical archives of 2 University Medical Centers in the Netherlands were used to collect 817 Formalin Fixed Paraffin Embedded (FFPE) liver biopsies, related to NASH and fibrosis. Samples of which there was suspicion of non-NASH-related fibrosis, e.g. Hepatitis B (HBV), Hepatitis C (HCV), Alcoholic Steatohepatitis (ASH) and Auto-immune Hepatitis (AIH) were excluded. In total 155 samples were included and pathological re-examination was performed by an independent pathologist. Scoring was performed according the SAF (Steatosis, Activity and Fibrosis) algorithm (PMID: 22707395; DOI: 10.1002/hep.25889). Both FFPE biopsies and matched serum samples from NASH patients with different fibrosis stages were used to determine blood-based biomarkers for NASH and fibrosis.

### FFPERNA Isolation and Sequencing

Total RNA was extracted from FFPE liver samples using glass beads and RNeasy FFPE kit (Qiagen). Special lysis and incubation conditions enabled reverse formaldehyde modifications of RNA. The lysis buffer efficiently released RNA from tissue sections while avoiding further RNA degradation. After removing ribosomal RNA, cDNA synthesis was performed (Qiagen). Thereafter, cDNA was ligated with the sequencing adapters and amplified by PCR. Quality and yield of the amplicon was measured (Fragment Analyzer, Agilent Technologies, Amstelveen, The Netherlands), DV200>40% was used as quality control for sufficient quality. Library prep was performed using NEBNext Ultra II Directional RNA Library Prep Kit for Illumina (NEB #E7760S/L). Clustering and DNA sequencing, using the Illumina NovaSeq6000, was performed according to manufacturer's protocols by service provider GenomeScan B.V (Leiden, the Netherlands), yielding 40-100 million sequencing clusters per sample and 2×150 bp Paired-End reads (PE) per cluster. The count data served as input for the statistical analysis using DEseq2 package (PMID: 25516281; doi:10.1186/s13059-014-0550-8). Selected differentially expressed genes (DEGs), corrected for multiple testing (available in DEseq2 package), were used for expression analyses.

### Steps Taken to Develop the Biomarker Panel

Various steps were taken to uncover the biomarker sets for diagnosis of NASH/fibrosis stage and fibrogenesis. In a preclinical animal study genes and proteins were identified which correlated with fibrosis and which could be detected at a time before there was manifestation of the disease itself (PMID: 29276754; DOI: 10.1016/j.jcmgh.2017.10.001). These mouse signature genes were translated into the human homologues.

RNA sequencing data from FFPE liver samples from NASH/fibrosis patients were used in which from the mouse signature dataset those genes were selected which were increased at a transcriptional level in patients with NASH/fibrosis vs non-fibrotic patients. The selected set of genes was further evaluated by selection for gene protein products which were produced in the liver. For this, literature and the human protein atlas were used to evaluate what the protein products of the genes of interest were, whether these protein products were produced in the liver, whether they were proteins present in the circulation and whether they could be detected in human plasma or serum. Furthermore, it was evaluated if the proteins were linked to fibrosis. Note that proteins that were NOT linked to liver or fibrosis were NOT immediately excluded from the list as they might represent novel biomarkers. This resulted in a set of potential circulating biomarker candidates, which was used for further evaluating in clinical serum samples.

### Measuring Biomarkers In Sera Using ELISA

The various biomarkers were analyzed in sera from NASH patients with different stages of fibrosis using commercial ELISA's. The serum samples used were from the same patients as used for FFPE gene expression data generation. The ELISA's were first validated by testing recovery and linearity of dilution. The ELISA's used were obtained from R&D Systems (Minneapolis, USA), Abbexa (Cambridge, UK), Cell Signalling Technology (Danvers, USA) and Millipore Sigma (St. Louis, USA). The ELISA's were applied according the manufacturer's instructions and using dilutions in the linear range of the ELISA. For 12 potential biomarker candidates a suitable assay was identified. They were analysed in sera from F0(n=4), F1 (n=2), F2 (n=10), F3 (n=9) and F4 (n=4).

### Using Algorithm to Predict Fibrosis Stage in NASH

Random forest classification is a machine learning method based on decision trees. In random forest algorithm, multiple deep decision trees are fitted to random sub-samples of the data. Here data is split not only by samples but also by features. Afterwards, predictions from multiple decision trees are averaged. Averaged predictions from multiple decision trees constitute the output of the random forest algorithm. Here, to use random forest classifier before predicting the fibrosis stages, we standardized the measured concentrations. To standardize the data, first we centered the concentrations of the biomarkers by making the mean of the measurement for each biomarker equal to zero. Then we divided the centered concentrations by the standard deviation for each biomarker. Therefore, for each measured biomarker, the mean value was equal to zero and standard deviation was equal to one. We used this data as input to random forest classifier algorithm.

### 2. Data Biomarkers Measured in Patient Sera; Diagnostic Set of Biomarkers

The 12 biomarkers candidates were used for analysis in sera of patients with various stages of fibrosis. The sera (n=29) were obtained from the same patients as used for FFPE gene expression analysis.

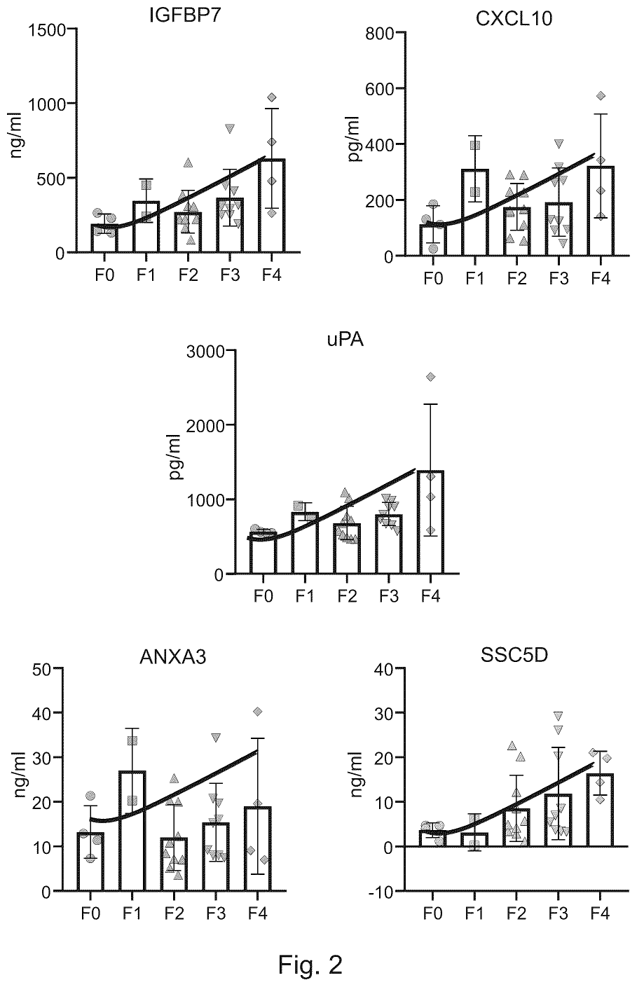

Table 2 and FIG. 2 clearly show for 5 of the biomarkers increased levels correlating with increased severity of fibrosis pathology. Table 1 shows data for all biomarkers.

TABLE 1
Biomarker analysis in sera from NASH/fibrosis patients with different stages of fibrosis.
THBS1 (ng/ml), PAM (ng/ml), VCAN (ng/ml), FBN1 (ng/ml), CXCL10 (pg/ml), ADAMTS2 (ng/ml),
IGFBP1 (ng/ml), TNC (pg/ml), SEMA4D (pg/ml), uPA (pg/ml), SSCD5 (ng/ml).

| | F0 | F1 | F2 | F3 | F4 |
|---|---|---|---|---|---|
| THBS1 | 57.3 ± 24.1 | 93.2 ± 7.0 | 88.1 ± 33.7 | 73.0 ± 25.7 | 34.1 ± 17.1 |
| PAM | 1.7 ± 0.6 | 2.2 ± 1.0 | 2.2 ± 0.5 | 1.8 ± 0.4 | 1.2 ± 0.3 |
| VCAN | 108.7 ± 31.1 | 181.0 ± 18.0 | 126.0 ± 30.8 | 119.0 ± 29.8 | 104.3 ± 28.5 |
| ANXA3 | 13.2 ± 5.9 | 27.0 ± 9.5 | 12.0 ± 7.4 | 15.4 ± 8.8 | 19.0 ± 15.2 |
| FBN1 | 34.8 ± 28.3 | 467.4 ± 28.2 | 48.3 ± 26.2 | 66.0 ± 44.7 | 56.5 ± 33.6 |
| CXCL10 | 113.7 ± 66.6 | 311.9 ± 118.4 | 175.1 ± 84.1 | 192.6 ± 12.3 | 322.9 ± 160.8 |
| ADAMTS2 | 17.6 ± 11.0 | 34.1 ± 2.3 | 25.4 ± 10.1 | 31.5 ± 20.5 | 25.9 ± 14.2 |
| IGFBP7 | 191.6 ± 65.5 | 346.4 ± 145.2 | 271.1 ± 143.3 | 366.6 ± 189.9 | 630.3 ± 290.4 |
| TNC | 17223.79 ± 8791.227 | 16867.2 ± 2475.157 | 19368.05 ± 11404.9 | 17595.83 ± 8816.083 | 31081.99 ± 23335.71 |
| SEMA4D | 650.3 ± 353.1 | 1069.3 ± 99.3 | 953.3 ± 623.7 | 618.1 ± 210.9 | 671.8 ± 234.2 |
| uPA | 566.7 ± 26.0 | 832.4 ± 120.2 | 680.2 ± 225.1 | 801.1 ± 154.4 | 1392.4 ± 884.9 |
| SSC5D | 3.7 ± 1.7 | 3.2 ± 4.2 | 8.6 ± 7.5 | 11.9 ± 10.4 | 16.4 ± 4.9 |

Selected biomarkers which increase with increasing fibrosis pathology, and which predict which fibrosis stage the patient has (correlation with pathology).

TABLE 2
Biomarker analysis in sera from NASH/fibrosis patients with different stages
of fibrosis. CXCL10 (pg/ml), IGFBP1 (ng/ml), uPA (pg/ml), SSCD5 (ng/ml).

| | F0 | F1 | F2 | F3 | F4 |
|---|---|---|---|---|---|
| IGFBP7 | 191.6 ± 65.5 | 346.4 ± 145.2 | 271.1 ± 143.3 | 366.6 ± 189.9 | 630.3 ± 290.4 |
| (ng/ml) | | | | | |
| CXCL10 | 113.7 ± 66.6 | 311.9 ± 118.4 | 175.1 ± 84.1 | 192.6 ± 12.3 | 322.9 ± 160.8 |
| uPA | 566.7 ± 26.0 | 832.4 ± 120.2 | 680.2 ± 225.1 | 801.1 ± 154.4 | 1392.4 ± 884.9 |
| ANXA3 | 13.2 ± 5.9 | 27.0 ± 9.5 | 12.0 ± 7.4 | 15.4 ± 8.8 | 19.0 ± 15.2 |
| SSC5D | 3.7 ± 1.7 | 3.2 ± 4.2 | 8.6 ± 7.5 | 11.9 ± 10.4 | 16.4 ± 4.9 |

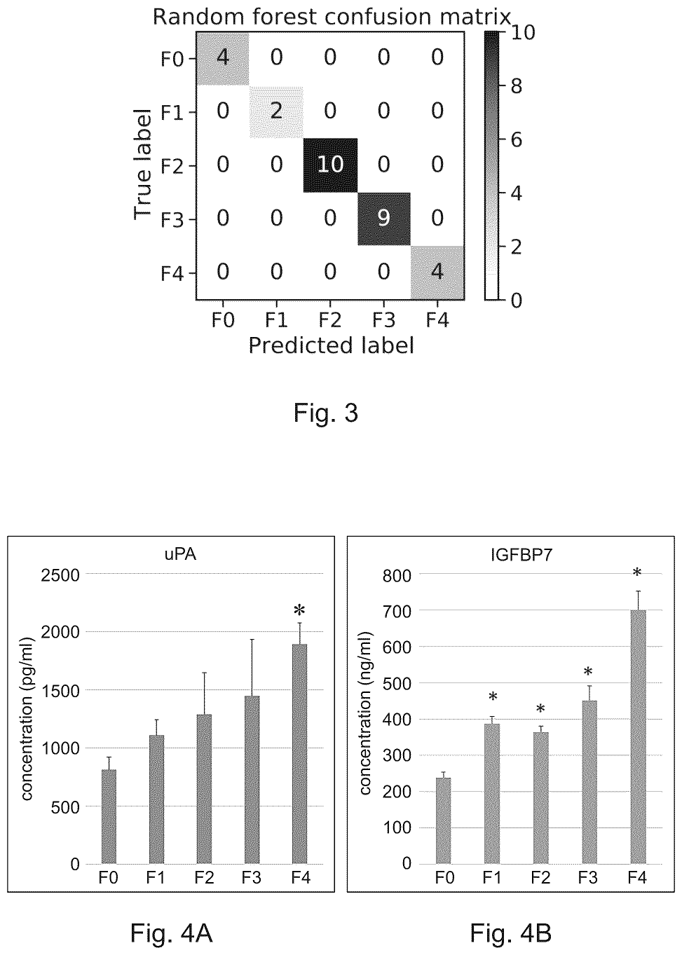

Using Random forest Confusion analysis, based on the serum data of 11 of the 12 biomarkers in 29 NASH patients with various stages of fibrosis (IGFBP7, THBS1, TNC, CXCL10, uPA, ANXA3, FBN1, VCAN, ADAMTS2, PAM and SSC5D), all patients were predicted for the right fibrosis stage (FIG. 3).

### 3. Verification of the Biomarkers in Larger Cohorts

To verify the relevance fibrosis staging 12 potential biomarkers were analyzed in a few cohorts of NAFLD patients with various levels of pathological assessed fibrosis staging. The concentration of a number of potential biomarkers were increased or decreased at increasing fibrosis stage.

Based on the biomarkers that are the most predictive for liver fibrosis, a prediction model for liver fibrosis staging was developed. The serum levels of potential biomarkers in the patient cohorts were divided into a training set and a test set (70/30). Stratified 5-fold cross validation (repeated 10 times) was applied to the training and test set. AUROC curve was used to show predictive value of a set biomarkers 1) to distinguish NAFLD fibrosis F-scores (F1-F4) compared to reference F0; and 2) to distinguish mild (F1-F2) and advanced (F3-F4) NAFLD fibrosis as compared to reference (F0).

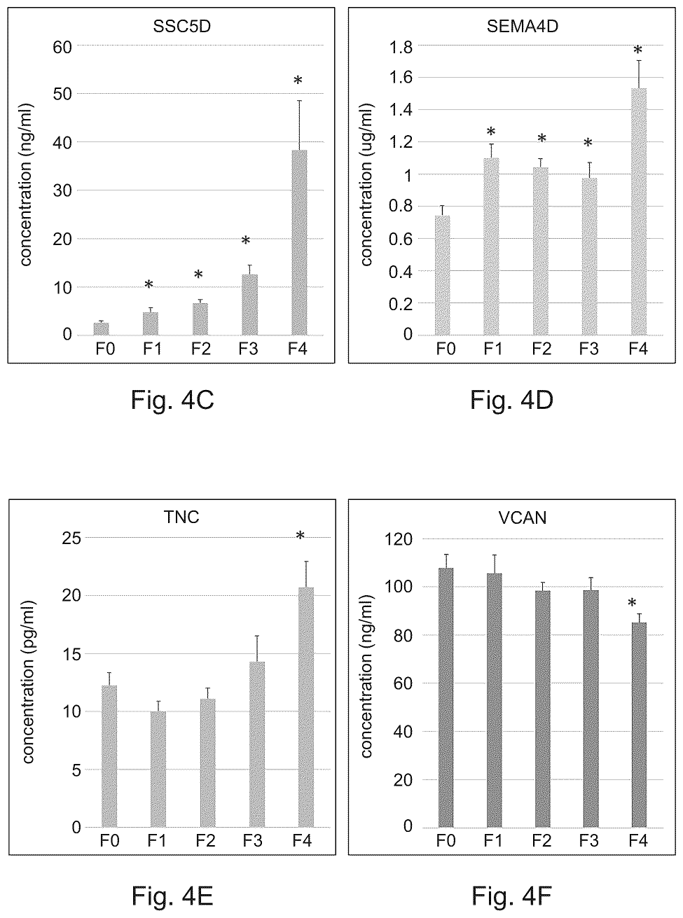

Protein levels of uPA (also referred to as urokinase_PLAU), IGFBP7, SSC5D, SEMA4D, TNC and VCAN were found to be most predictive. FIG. 4 shows the serum protein levels of these proteins in samples from NASH patients with biopsy-proven fibrosis score (F0-F4).

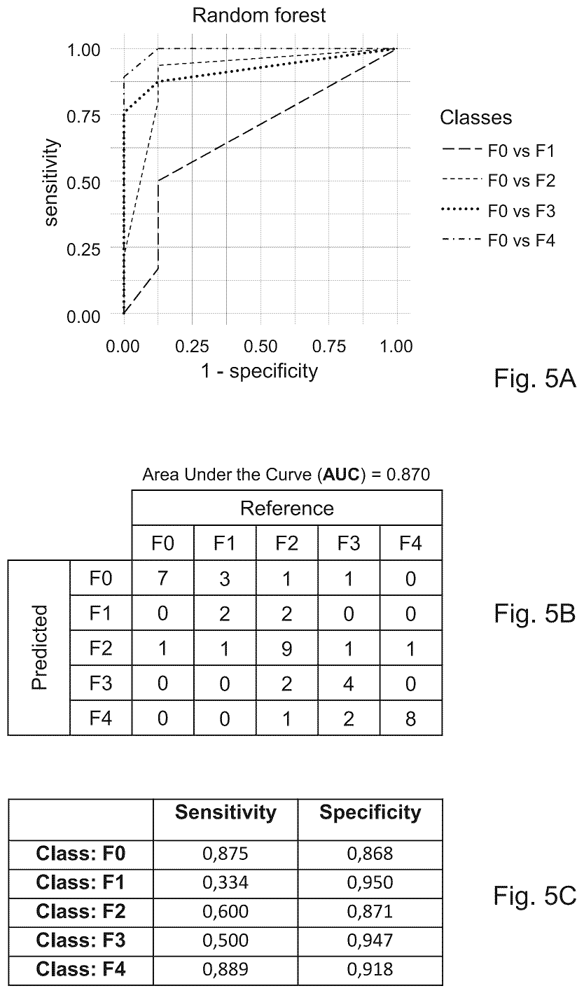

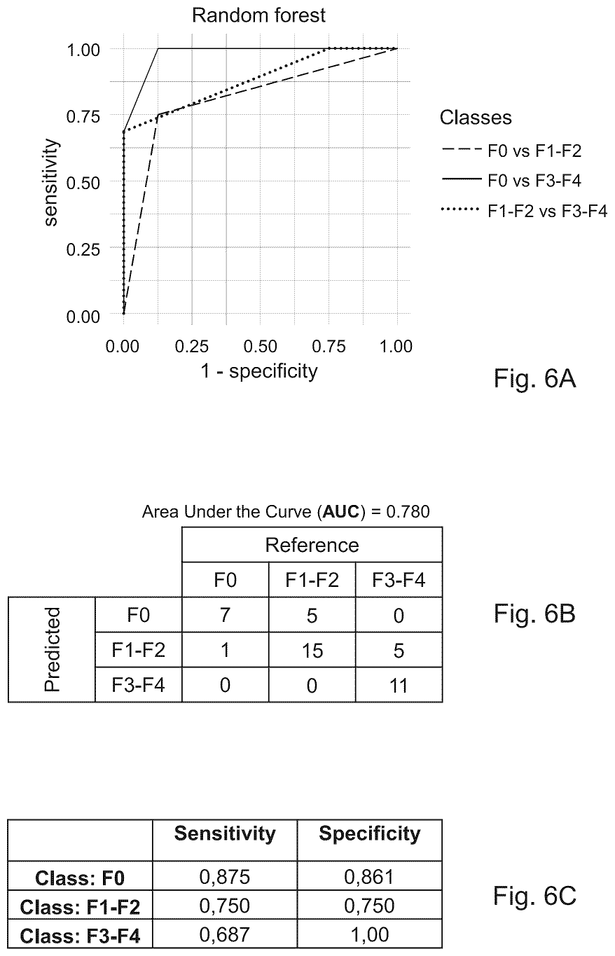

FIGS. 5 and 6 show the predictive value using serum data from IGFBP7, SSC5D and SEMA4D, as example, to distinguish the individual NAFLD fibrosis F-scores (F1-F2-F3-F4) as compared to reference F0(FIG. 5) and to distinguish the grouped F-scores (mild, F1 and F2; advanced, F3 and F4) as compared to reference F0(FIG. 6). The area under the curve represents a combined value for sensitivity and specificity, whereby a value of above 0.65 is considered to represent a particularly good combination of sensitivity and specificity.

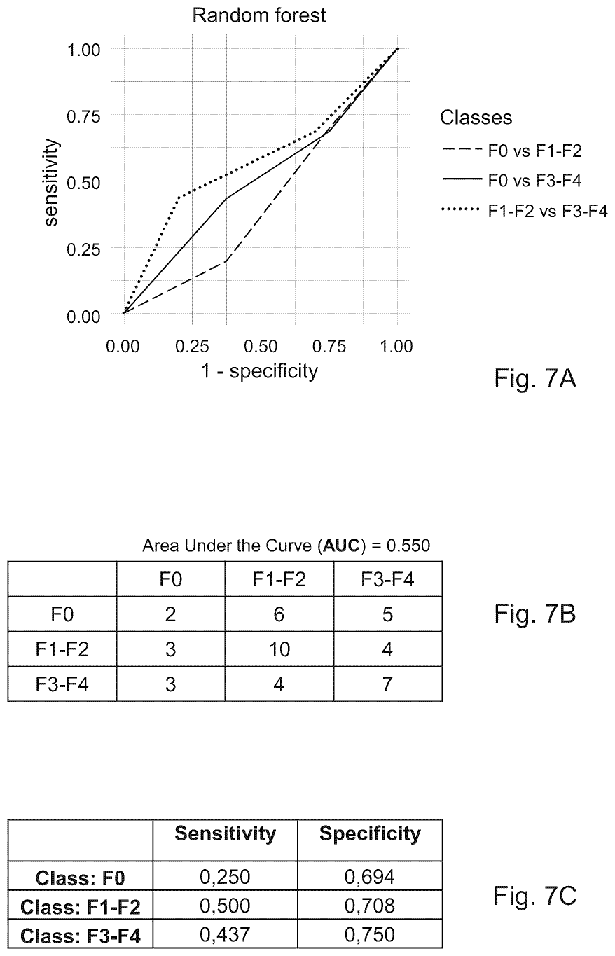

FIG. 7 shows the predictive value to distinguish the grouped F-scores (mild, F1 and F2; advanced, F3 and F4) as compared to reference F0using serum data from FBN1, VCAN and THBS1 for comparison.

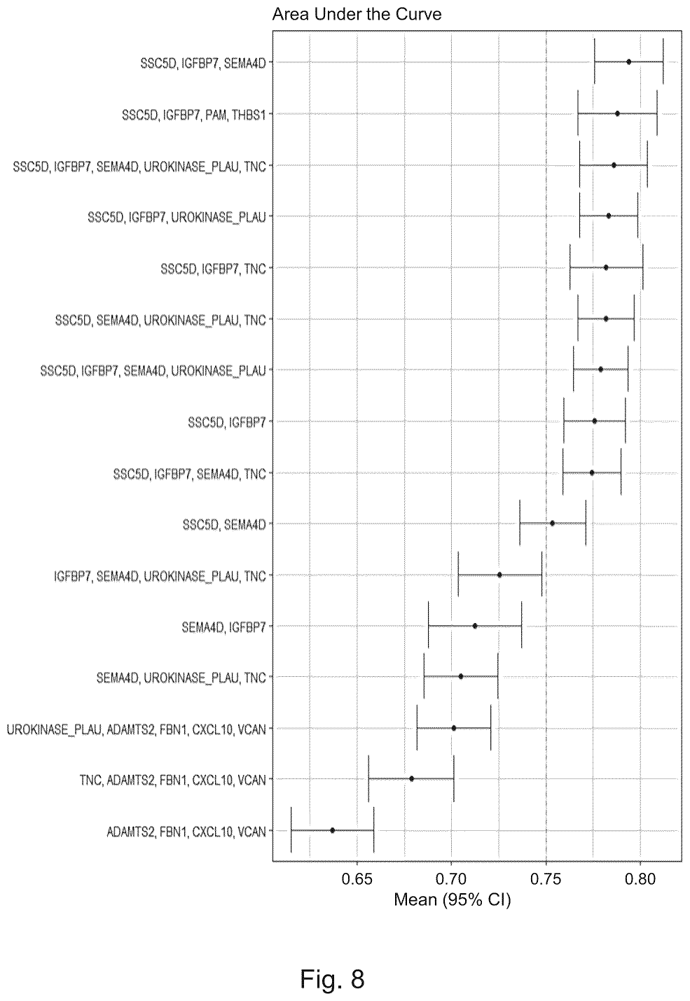

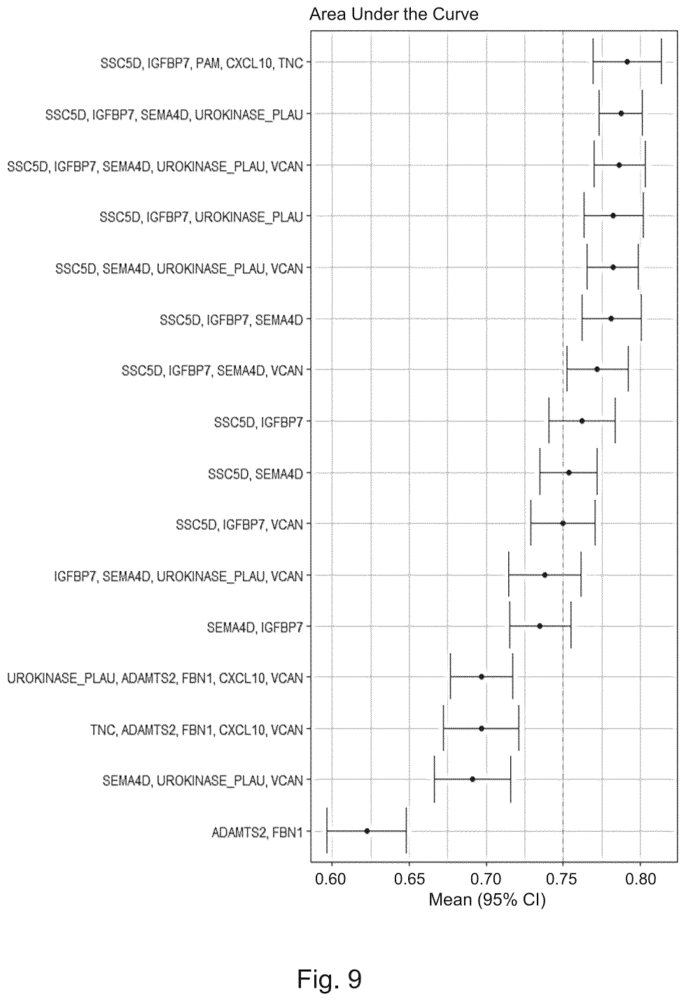

Assessing the predictive value of several different combinations of protein biomarkers confirms the predictive value of protein biomarkers profiles containing at least one of uPA, IGFBP7, SSC5D, SEMA4D and VCAN both to distinguish individual NAFLD fibrosis F-scores (F1-F2-F3-F4) and grouped F-scores (mild, F1 and F2; advanced, F3 and F4) as compared to reference F0(see FIGS. 8 and 9, respectively). As is further apparent from these figures, the predictive value of protein biomarkers profiles containing one, two or three of IGFBP7, SSC5D and SEMA4D has a particular good predictive value with particularly high area under the curve, i.e. a combination of a high sensitivity and high specificity.

### REFERENCES

Cusi, K.; Chang, Z.; Harrison, S.; Lomonaco, R.; Bril, F.; Orsak, B.; Ortiz-Lopez, C.; Hecht, J.; Feldstein, A.E.; Webb, A.; et al. Limited value of plasma cytokeratin-18 as a biomarker for NASH and fibrosis in patients with non-alcoholic fatty liver disease. J. Hepatol. 2014, 60, 167-174.
Kazankov, K.; Barrera, F.; Jon Møller, H.; Chiara Rosso, C.; Bugianesi, E.; David, E.; Ibrahim Kamal Jouness, R.; Esmaili, S.; Eslam, M.; McLeod, D.; et al. The macrophage activation marker sCD163 is associated with morphological disease stages patients with non-alcoholic fatty liver disease. Liver Int. 2016, 36, 1549-1557.
van Koppen A, Verschuren L, van den Hoek AM, Verheij J, Morrison MC, Li K, Nagabukuro H, Costessi A, Caspers MPM, van den Broek TJ, Sagartz J, Kluft C, Beysen C, Emson C, van Gool AJ, Goldschmeding R, Stoop R, Bobeldijk-Pastorova I, Turner SM, Hanauer G, Hanemaaijer R. Uncovering a Predictive Molecular Signature for the Onset of NASH-Related Fibrosis in a Translational NASH Mouse Model. Cell Mol Gastroenterol Hepatol. 2017 Oct 14;5(1):83-98.e10. doi: 10.1016/j.jcmgh.2017.10.001.
Loomba, R.; Jain, A.; Diehl, A.M.; Guy, C.D.; Portenier, D.; Sudan, R.; Singh, S.; Faulkner, C.; Richards, L.; Hester, K.D.; et al. Validation of Serum Test for Advanced Liver Fibrosis in Patients with Nonalcoholic Steatohepatitis. Clin. Gastroenterol. Hepatol. 2019, 17, 1867-1876.
Palekar, N.A.; Naus, R.; Larson, S.P.; Ward, J.; Harrison, S.A. Clinical model for distinguishing nonalcoholic steatohepatitis from simple steatosis in patients with nonalcoholic fatty liver disease. Liver Int. 2006, 26, 151-156.
Ratziu V.; Friedman S. L. Why do so many NASH trials fail? Gastroenterology. 2020 May 18;S0016-5085(20)30680-6. doi: 10.1053/j.gastro.2020.05.046.
Kleiner DE, Brunt EM, Van Natta M, Behling C, Contos MJ, Cummings OW, Ferrell LD, Liu YC. Torbenson MS, Unalp-Arida A, Yeh M, Mccullough AJ, Sanyal AJ: Nonalcoholic Steatohepatitis Clinical Research Network. Design and validation of a histological scoring system for nonalcoholic fatty liver disease. Hepatology. 2005 Jun;41(6): 1313-21. doi: 10.1002/hep.20701. PMID: 15915461.

1-24. (canceled)

25. A kit of parts comprising means for determining protein level of at least two proteins, wherein one of said at least two proteins is selected from Insulin-like growth factor-binding protein 7 (IGFBP7), Urokinase-type plasminogen activator (uPA), Semaphorin 4D (SEMA4D), Versican (VCAN), and Scavenger Receptor Cysteine Rich Family Member With 5 Domains (SSC5D), and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

26. The kit of parts according to claim 25, wherein one of said at least two proteins is selected from IGFBP7, SEMA4D, and SSC5D, and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

27. (canceled)

28. The kit of parts according to claim 25, wherein said at least two proteins are selected from IGFBP7, SEMA4D, and SSC5D.

29. (canceled)

30. A method for analysing a blood, serum, or plasma sample of a subject, the method comprising determining the protein level of at least two proteins, wherein one of said at least two proteins is selected from Insulin-like growth factor-binding protein 7 (IGFBP7), Urokinase-type plasminogen activator (uPA), Semaphorin 4D (SEMA4D), Versican (VCAN), and Scavenger Receptor Cysteine Rich Family Member With 5 Domains (SSC5D), and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

31. The method according to claim 30, wherein said subject is suffering from fatty liver disease, in particular from non-alcoholic fatty liver disease (NAFLD), or non-alcoholic steatohepatitis (NASH), or suspected of suffering therefrom.

32. The method according to claim 30, wherein one of said at least two proteins is selected from IGFBP7, SEMA4D, and SSC5D, and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

33. The method according to claim 30, wherein one of said at least two proteins is selected from SEMA4D and SSC5D, and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

34. The method according to claim 30, comprising determining the protein level of at least two proteins selected from IGFBP7, SEMA4D, and SSC5D.

35. The method according to claim 30, comprising determining the protein levels of at least SEMA4D and SSC5D.

36. The method according to claim 30, further comprising determining the protein level of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10), and Annexin A3 (ANXA3).

37. The method according to claim 30, comprising determining the protein levels of IGFBP7, SEMA4D, and SSC5D; IGFBP7, TNC, and SSC5D; SEMA4D, uPA, and TNC; IGFBP7, SSC5D, and uPa; IGFBP7, SSC5D, and TNC; IGFPB7, SSC5D, SEMA4D, and uPA; SEMA4D, SSC5D, uPA, and TNC; IGFBP7, SEMA4D, SSC5D, and TNC; or IGFBP7, SEMA4D, uPA, and TNC.

38. The method according to claim 30, wherein the protein levels are compared with a reference or a value for the protein levels is compared with a reference value.

39. The method according to claim 38 wherein said reference value is the protein levels of the same at least two proteins in a blood, serum, or plasma sample, or a value for said protein levels, from a healthy subject or subjects, from a subject or subjects suffering from NAFLD, or from a subject or subjects suffering from NASH, or from a subject or subjects suffering from NASH fibrosis stage F0, F1, F2, F3 or F4.

40. The method of claim 30, further comprising classifying the subject, the method comprising:
comparing said determined protein levels or a value for said protein levels with a reference value, said reference value being the protein levels of the same proteins or a value for said protein levels in a blood, serum, or plasma sample from healthy subjects, from subjects suffering from non-alcoholic fatty liver disease (NAFLD), or from subjects suffering from NASH, in particular from subjects suffering from NASH fibrosis stage F0, F1, F2, F3 or F4,
classifying the subject as suffering from NAFLD, NASH, or hepatic fibrosis and/or determining NASH fibrotic stage.

41. The method of claim 30, further comprising classifying and treating the subject, the method comprising:
comparing said determined protein levels or a value for said protein levels with a reference value, said reference value being the protein levels of the same proteins or a value for said protein levels in a blood, serum, or plasma sample from healthy subjects, from subjects suffering from non-alcoholic fatty liver disease (NAFLD), or from subjects suffering from NASH, in particular from subjects suffering from NASH fibrosis stage F0, F1, F2, F3 or F4,
classifying the subject as suffering from NAFLD, NASH, or hepatic fibrosis and/or determining NASH fibrotic stage; and
treating the subject classified as suffering from NAFLD, NASH, or hepatic fibrosis and/or a particular NASH fibrotic stage with liver transplantation or with active agents.

42. A method for assigning subjects to a clinical trial for treatment or prevention of non-alcoholic steatohepatitis (NASH), the method comprising assigning subjects that are classified as suffering from NAFLD, NASH, or hepatic fibrosis and/or determining NASH fibrotic stage to said clinical trial, wherein said subjects have been classified by determining the protein level of at least two proteins in a blood, serum, or plasma sample of said subject, wherein one of said at least two proteins is selected from Insulin-like growth factor-binding protein 7 (IGFBP7), Urokinase-type plasminogen activator (uPA), Semaphorin 4D (SEMA4D), Versican (VCAN), and Scavenger Receptor Cysteine Rich Family Member With 5 Domains (SSC5D), and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2), and wherein said at least two proteins are different.

43. The method according to claim 42, wherein one of said at least two proteins is selected from IGFBP7, SEMA4D, and SSC5D, and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

44. The method according to claim 42, wherein one of said at least two proteins is selected from SEMA4D and SSC5D, and the other of said at least two proteins is selected from IGFBP7, TNC, uPA, SEMA4D, VCAN, SSC5D, Protein Associated with MYC (PAM), and A disintegrin and metalloproteinase with thrombospondin motifs 2 (ADAMTS2).

45. The method according to claim 42, comprising determining the protein level of at least two proteins selected from IGFBP7, SEMA4D, and SSC5D or the protein levels of at least SEMA4D and SSC5D.

46. The method according to claim 42, further comprising determining the protein level of one or more further proteins selected from the group consisting of IGFBP7, TNC, uPA, SEMA4D, VCAN, SSC5D, PAM, ADAMTS2, Thrombospondin 1 (THBS1), C-X-C motif chemokine ligand 10 (CXCL10), and Annexin A3 (ANXA3).
